(19) United States
(12) Patent Application Publication
(10) Pub. No.: US 2025/0268499 A1
(43) Pub. Date: Aug. 28, 2025

(54) Systems and Methods for Predicting Behavioral Traits

(71) Applicant: Georgia State University Research Foundation, Inc., Atlanta, GA (US)

(72) Inventors: Vince Calhoun, Decatur, GA (US); Jing Sui, Atlanta, GA (US); Shile Qi, Atlanta, GA (US)

(21) Appl. No.: 18/551,277

(22) PCT Filed: Jan. 20, 2022
(86) PCT No.: PCT/US2022/013117

§ 371 Date: Sep. 19, 2023

Related U.S. Application Data
(60) Provisional application No. 63/163,862, filed on Mar. 20, 2021.

(51) Int. Cl. A61B 5/16; A61B 5/00; A61B 5/055; G16H 50/30

(52) U.S. Cl. CPC A61B 5/168; A61B 5/055; A61B 5/165; A61B 5/4064; G16H 50/30

(57) ABSTRACT

Systems and methods for evaluating the mental health of a patient by identifying multimodal reward related biomarkers for novelty seeking, or similar personal traits, associated with functional and structural alterations in adolescent brains. For example, a method may comprise identifying a highest portion of novelty seeking associated multimodal brain networks in a first cohort of persons based on Magnetic Resonance Imaging data, computing a plurality of scores, each score indicating a risk of developing one of a plurality of specified behaviors, determining, for the first cohort of persons at a later time, how accurately the computed plurality of scores indicated the risk of developing each of the plurality of specified behaviors, classifying at least a second person not belonging to the first cohort of persons using features of those novelty seeking networks, and displaying to a user the risk of the second person of developing any of the specified behaviors.

### STATEMENT REGARDING FEDERALLY SPONSORED RESEARCH OR DEVELOPMENT

This invention was made with Government support under R01MH117107 awarded by the National Institutes of Health. The Government has certain rights in the invention.

### FIELD OF THE INVENTION

The invention is generally directed to systems and methods for generating predictive biomarkers from structural and functional imaging data and associated clinical data. More particularly, the invention presents multimodal reward related biomarkers associated with functional and structural alterations in the brain for predicting high-novelty seeking behavior or similar behavioral traits in adolescents. Furthermore, these biomarkers can be used to predict disease risk or severity of many different mental health disorders, including substance use disorders, hyperactivity, depression, and psychosis.

### BACKGROUND OF THE INVENTION

Adolescence is a critical development period in which personalities and behavioral tendencies that extend into personal adulthood are established. One of the largest behavioral changes observed in adolescence is risk taking and novelty seeking (NS), which are highly correlated. Novelty seeking assesses preference for seeking novel experiences and higher levels of rewarding stimulation, which is a hallmark of typical adolescent behavior, as well as a key personality trait in adolescents that is of critical importance in transitioning from a dependent child to an independent adult. Therefore, the adolescents with NS outlier scores might be related with risks towards developmental disorders. Coincidently, recent studies show that adolescent smokers, and problematic drug users both exhibit significantly higher NS scores than normal controls. Also, adolescents show more inclination to risk taking and NS, possibly with a hypersensitivity to reward.

On the other hand, reward processing is impaired in multiple disorders, including substance use disorders, depression and psychosis (including negative symptoms). The changes of mechanisms underlying reward processing are important for NS behavior, and are also to likely influence depression and psychosis, albeit in a different way. Nonetheless, there could still be core properties of reward processing that remain invariant to modulate distinct psychopathologies. Although there are links between NS and reward circuitry dysfunction through dopamine modulation, and between reward circuitry dysfunction and multiple disorders, the specific associations and longitudinal impact among NS, reward circuitry dysfunction and risks for multiple disorders (including drinking, smoking, attention-deficit/hyperactivity disorder (ADHD), depression and psychosis) remain unexplored and quantitative tools for assessing these associations have not been available.

“Multimodal” is a widely used phrase in the context of brain imaging studies. Collecting multiple modalities of magnetic resonance imaging (MRI) data from the same individual has become popular in brain imaging studies. There is increasing evidence that multimodal brain imaging studies can help provide a more complete understanding of the brain and its disorders, for example it can inform us about how brain structure shapes brain function, in which way they are impacted by psychopathology and which functional or structural aspects of physiology could drive human behavior and cognition.

It is becoming increasingly clear that combining multi-modal brain imaging data is able to provide more information for individual subjects by exploiting the rich multimodal information that exists. However, the number of studies that do true multimodal fusion (i.e. capitalizing on joint information among modalities) is still remarkably small given the known benefits. In part, this is because multi-modal studies require broader expertise in collecting, analyzing, and interpreting the results than do unimodal studies.

IMAGEN is a European research project examining how biological, psychological, and environmental factors during adolescence may influence brain development and mental health. IMAGEN provides a dataset from a large cross-sectional European multicenter study of reinforcement sensitivity in adolescents. Many IMAGEN studies have identified risk biomarkers in healthy adolescents for individual disorders like smoking, alcohol use, drug abuse, ADHD, depression and psychosis-like experience, but mostly within one modality or without exploring imaging predictors of symptoms severity in multiple external patient cohorts.

Thus, there is a need for systems and methods to identify multimodal reward related biomarkers associated with high-NS in adolescents that will predict developmental risks of mental illnesses.

### SUMMARY OF THE INVENTION

The present invention provides systems and methods for evaluating and treating the mental health of a patient by identifying multimodal reward related biomarkers for novelty seeking, or similar personal traits, associated with functional and structural alterations in adolescent brains. The invention provides multimodal reward related biomarkers, including the thalamus, the prefrontal cortex, the insular cortex, the mid temporal lobe, the striatum, the amygdala, and the hippocampus, associated with high-NS in adolescents at age 14. The prediction models built on these biomarkers are able to longitudinally predict five different risk scales including alcohol drinking, smoking, hyperactivity, depression, and psychosis for the same and unseen adolescents at age 19, and even predict the corresponding symptom scores of five types of patients across cohorts. Furthermore, the identified reward-related multimodal features can classify among ADHD, MDD and SZ with an accuracy of 87.2%.

For example, in an embodiment, a method may be implemented in a computer system comprising a processor, memory accessible by the processor, and computer program instructions stored in the memory and executable by the processor and the method may comprise identifying a highest portion of novelty seeking associated multimodal brain networks in a first cohort of persons based on Magnetic Resonance Imaging data, computing a plurality of scores, each score indicating a risk of developing one of a plurality of specified behaviors, determining, for the first cohort of persons at a later time, how accurately the computed plurality of scores indicated the risk of developing each of the plurality of specified behaviors, classifying at least a second person using features of those novelty seeking associated multimodal brain networks that indicated the risk of developing the specified behaviors with greater than a predefined accuracy, and displaying to a user the risk of the second person of developing any of the plurality of specified behaviors.

In embodiments, the novelty seeking associated multimodal brain networks may comprise at least one of the thalamus, the prefrontal cortex, the insular cortex, the mid temporal lobe, the striatum, the amygdala, and the hippocampus. The plurality of specified behaviors may comprise at least some of alcohol drinking, smoking, hyperactivity, depression, and psychosis. The Magnetic Resonance Imaging data may comprise a multiple MRI fusion, including gray matter volume (GMV) and plurality of task-related fMRI contrasts. The plurality of task-related fMRI contrasts may comprise at least some of a modified monetary incentive delay task, a face emotion identification task, and a stop-signal task. Combining the features of those novelty seeking associated multimodal brain networks that indicated the risk of developing the specified behaviors with greater than a predefined accuracy may be used to form a model of a generalized dysfunctional novelty seeking associated multimodal brain network. Classifying the risk of developing any of the plurality of specified behaviors may comprise at least one of: a regression model, a machine learning model, an image analysis, a support vector machine, a binary classification, or a multi-class classification.

In an embodiment, a system may comprise a processor, memory accessible by the processor, and computer program instructions stored in the memory and executable by the processor to perform identifying a highest portion of novelty seeking associated multimodal brain networks in a first cohort of persons based on Magnetic Resonance Imaging data, computing a plurality of scores, each score indicating a risk of developing one of a plurality of specified behaviors, determining, for the first cohort of persons at a later time, how accurately the computed plurality of scores indicated the risk of developing each of the plurality of specified behaviors, classifying at least a second person using features of those novelty seeking associated multimodal brain networks that indicated the risk of developing the specified behaviors with greater than a predefined accuracy, and displaying to a user the risk of the second person of developing any of the specified behaviors.

In an embodiment, a computer program product may comprise a non-transitory computer readable storage having program instructions embodied therewith, the program instructions executable by a computer, to cause the computer to perform a method that may comprise identifying a highest portion of novelty seeking associated multimodal brain networks in a first cohort of persons based on Magnetic Resonance Imaging data, computing a plurality of scores, each score indicating a risk of developing one of a plurality of specified behaviors, determining, for the first cohort of persons at a later time, how accurately the computed plurality of scores indicated the risk of developing each of the plurality of specified behaviors, classifying at least a second person using features of those novelty seeking associated multimodal brain networks that indicated the risk of developing the specified behaviors with greater than a predefined accuracy, and displaying to a user the risk of the second person of developing any of the specified behaviors.

In an embodiment, the method may comprise identifying a highest portion of novelty seeking associated multimodal brain networks in a first cohort of persons based on Magnetic Resonance Imaging data. The method may further comprise computing a plurality of scores, each score indicating a risk of developing one of a plurality of specified behaviors followed by determining, for the first cohort of persons at a later time, how accurately the computed plurality of scores indicated the risk of developing each of the plurality of specified behaviors. Then the method may build a predictive model able to classify a second person's risk of developing at least one of the plurality of specified behaviors based on at least magnetic resonance imaging data of the second person.

In an embodiment, the method may comprise measuring a person's brain using magnetic resonance imaging and feeding the measurements into a predictive model. The predictive model may determine a person's risk of developing one or more of a plurality of specified behaviors. The method may then display the risk that the person is likely to develop one or more of the plurality of specified behaviors.

### BRIEF DESCRIPTION OF THE DRAWINGS

The details of the present invention can best be understood by referring to the accompanying drawings, in which like reference numbers and designations refer to like elements.

FIG. 1 illustrates an overview of the study design according to embodiments of the present invention.

FIG. 2 is the analysis flowchart according to embodiments of the present invention.

FIGS. 3A and 3B are spatial brain maps and correlation scatter plots showing the identified high-NS associated multimodal components in 14-year-olds subset.

FIGS. 4A-4E show spatial brain maps and prediction results based on the identified high-NS associated multimodal brain imaging networks.

FIG. 5A-5D are ROC curves and confusion matrixes obtained from classification based on the identified features.

FIG. 6 shows the permutation test for the correlation analysis between GM_IC1 and impulsiveness scores (10000 times).

FIGS. 7A-7C are spatial brain maps and correlation scatter plots showing the identified high-NS associated multimodal components in 14 years old subset.

FIGS. 8A and 8B show the most frequently occurring voxels associated with the permuted NS scores.

FIG. 9 shows the prediction results after regressing out gender.

FIG. 10 shows the prediction results of the Spearman correlation.

FIG. 11 shows the comparison of the contributing weights for 4 imaging features: reward task, emotion task, inhibition task and GM in prediction models.

FIGS. 12A-12D show the null distribution for the classification accuracies.

FIG. 13 is an illustration of the overlay all the identified high-NS-associated brain regions across 4 modalities.

FIGS. 14A-14E show prediction results after removing the emotion task.

FIGS. 15A-15E show prediction results after removing the inhibition control task.

FIG. 16 shows an exemplary block diagram of a computer system in which processes and embodiments of the invention may be implemented.

### DETAILED DESCRIPTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Methods for Identifying Multimodal Reward Related Biomarkers Associated with High-NS

Recently, a meta-analysis shows that the personality trait is significantly correlated with almost every psychiatric disorder, including ADHD, bipolar disorder, MDD, and SZ, based on heritability within the general population (Brainstorm, et al., Science, 360(6395): eaap8757(2018)). Coincidently, symptomatic and epidemiological comorbidity (Barch, et al., World Psychiatry. 13:224-232(2014), Qi, et al., Translational psychiatry. 10:149(2020), Sha, et al., Biological psychiatry. 85:379-388(2019), Plana-Ripoll, et al., JAMA psychiatry. 76:259-270(2019)) has also been reported for these mental disorders including bipolar disorder (Hirschfeld, et al., The Journal of clinical psychiatry. 64:53-59(2003)), MDD (Pan, et al., Jama. 306:1241-1249(2011)), autism spectrum disorder and ADHD. Considering the well-validated linkage between NS and reward processing, the present invention relates to high NS-associated multimodal brain networks in adolescents which represent a common dysfunctional circuit among smoking, drinking, hyperactivity, depression and psychosis, and these features are able to predict disease risk longitudinally in the follow up adolescents and external patient cohorts.

As known, NS depends on reinforcement through external or internal (drug experience, but perhaps also prayer/meditation) novel stimuli. The present invention uses the structural and functional brain mechanisms that underlie the individual cognitive components of reinforcement in relation to NS, namely reward processing, impulsiveness and emotional processing, that play an essential role in forming personality differences during adolescent development, to help diagnose many neuropsychiatric disorders, including addiction, ADHD, SZ and depression. As an example, the IMAGEN dataset was used as a discovery cohort, which is a large cross-sectional European multicenter study of reinforcement sensitivity in adolescents. Many IMAGEN studies have identified risk biomarkers in healthy adolescents for individual disorders like smoking, alcohol use, drug abuse, ADHD, depression and psychosis-like experience, but mostly within one modality or without exploring imaging predictors of symptoms severity in multiple external patient cohorts.

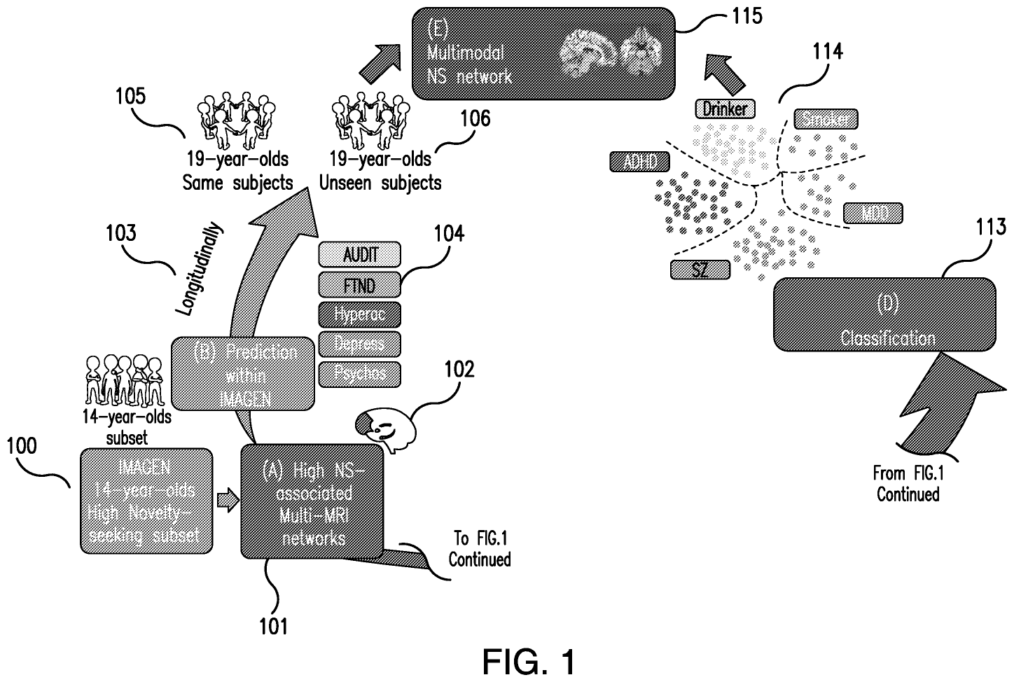

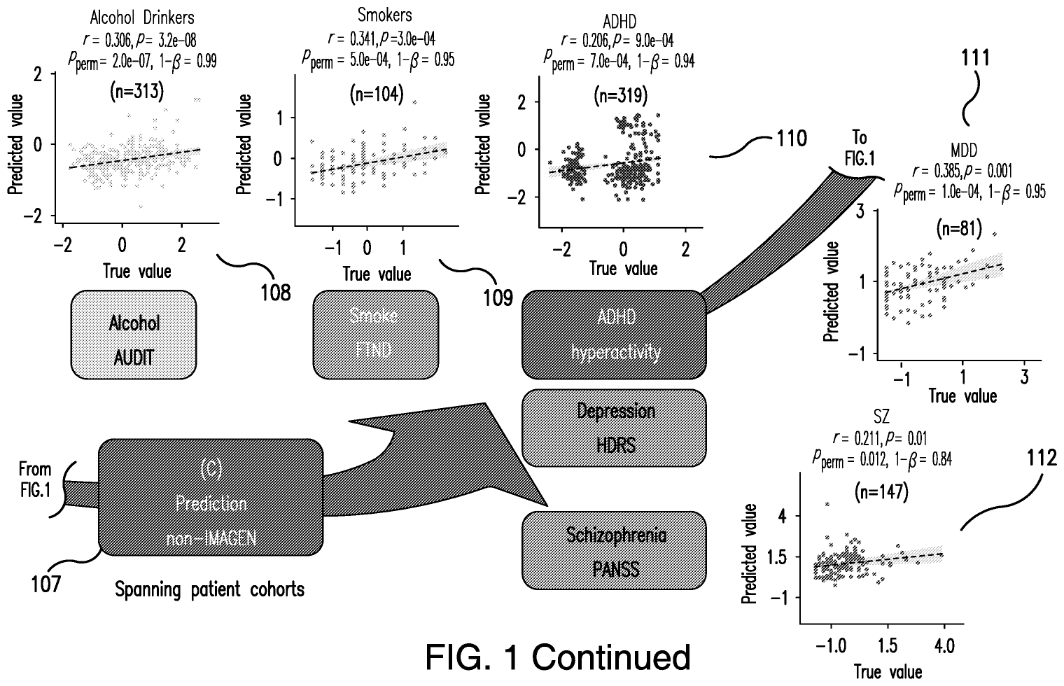

The study under which the invention was conceived, was designed to: 1) Identify high-NS associated multi-MRI networks (101) on the top 20% NS scored adolescents at 14-years old (FIG. 1A, 239 out of 1378, IMAGEN 14-year olds high novelty-seeking subset (100)). 2) Follow up the IMAGEN study to evaluate whether the identified high NS-associated multimodal features (102) can longitudinally predict (103) five different risk scores (104) for the same 19-year old subjects (105) (n=239), and the unseen youth (n=1100) at the age of 19 (106) (FIG. 1B). 3) Verify the external patient cohort (n=965) (107) to determine whether high-NS associated features (102) can predict symptom scores for alcohol drinking (AUDIT) (108), smoking (FTND) (109), ADHD (hyperactivity) (110), MDD (depression) (111) and SZ (psychosis) (112) (FIG. 1C). 4) Use the high-NS associated multimodal features (102) for classification (113), between patients (n=965) and controls (n=1094) as well as among different patient groups (114) (FIG. 1D). 5) Provide a generalized dysfunctional multimodal NS network (115) spanning alcohol (108, smoking (109), hyperactivity (110), depression (111), and psychosis (112) (FIG. 1E). The present invention goes beyond predicting the development of addiction and other mental health disorders, a worthwhile goal in itself for clinicians; it further allows the examination and refining of the neural basis common or specific to various psychiatric disorders.

Details of the experimental procedures are provided in the Examples section which follows. Briefly, NS scores were used as a reference to guide a four-way MRI fusion, including gray matter volume (GMV) and three task-related fMRI contrasts, to identify the high-NS (top 20% scored) associated brain regions in 14-year-olds adolescents. This supervised fusion model, MCCAR+JICA (multi-site canonical correlation analysis with reference+joint independent component analysis) can identify multimodal imaging components associated with a specific measure of interest (NS). The three representative fMRI tasks are: 1) modified monetary incentive delay, MID (anticipation of large gain vs no gain), which relates to reward anticipation; 2) faces task (angry vs control) that relates to emotional processing and 3) stop-signal task (stop failure vs baseline) that relates to reinforcement in adolescent. The identified NS-associated multimodal features in 14-year-olds were then used to build prediction models for both follow up (19-year-olds) disease risks prediction and transdiagnostic symptom severity evaluation, and even classification among multiple diseases.

The present invention relates to systems and methods for identifying multimodal reward related biomarkers associated with high-NS in adolescents that will predict developmental risks of mental illnesses. The present invention may use biomarkers to train a predictive model to estimate the risk that a patient may develop such behavioral conditions at an older age. In addition, the present invention may display such risks or calculated scores to aid a clinician in diagnosing such conditions. The clinician may also be shown the details of the training cohort or cohorts used to train the predictive model as well as details of the predictive model.

### II. Systems for Identifying Multimodal Reward Related Biomarkers Associated with High-NS

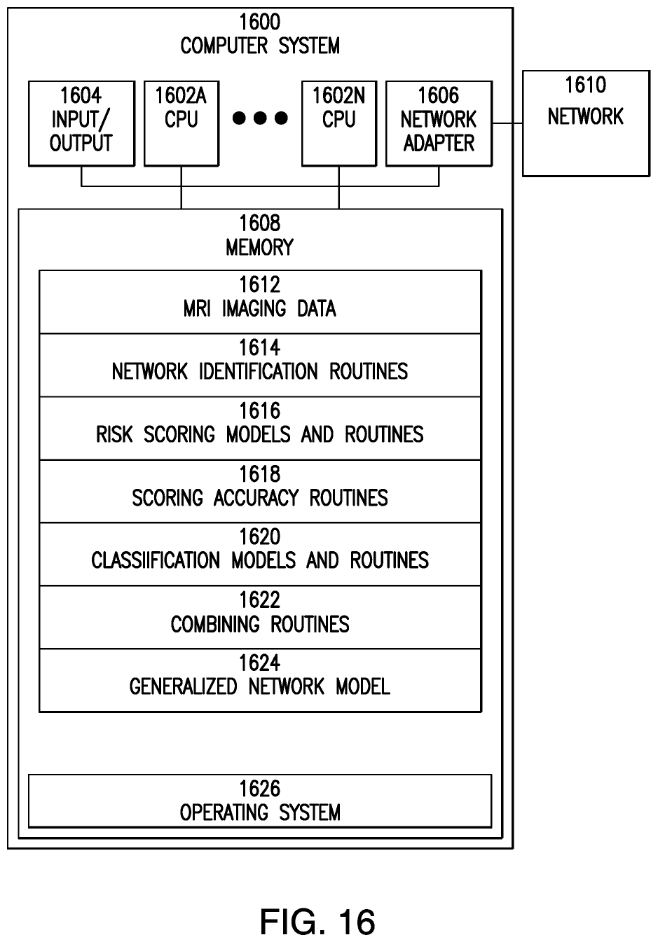

An exemplary block diagram of a computer system 1600, in which processes and components involved in the embodiments described herein may be implemented, is shown in FIG. 16. Computer system 1600 may be implemented using one or more programmed general-purpose computer systems, such as embedded processors, systems on a chip, personal computers, workstations, server systems, and minicomputers or mainframe computers, or in distributed, networked computing environments such as the cloud. Computer system 1600 may include one or more processors (CPUs) 1602A-1602N, input/output circuitry 1604, network adapter 1606, and memory 1608. CPUs 1602A-1602N execute program instructions in order to carry out the functions of the present communications systems and methods. Typically, CPUs 1602A-1602N are one or more microprocessors, such as an INTEL CORE® processor. FIG. 16 illustrates an embodiment in which computer system 1600 is implemented as a single multi-processor computer system, in which multiple processors 1602A-1602N share system resources, such as memory 1608, input/output circuitry 1604, and network adapter 1606. However, the present communications systems and methods also include embodiments in which computer system 1600 is implemented as a plurality of networked computer systems, which may be single-processor computer systems, multi-processor computer systems, or a mix thereof.

Input/output circuitry 1604 provides the capability to input data to, or output data from, computer system 1600. For example, input/output circuitry may include input devices, such as keyboards, mice, touchpads, trackballs, scanners, analog to digital converters, etc., output devices, such as video adapters, monitors, displays, printers, etc., and input/output devices, such as, modems, etc. Network adapter 1606 interfaces device 1600 with a network 1610. Network 1610 may be any public or proprietary LAN or WAN, including, but not limited to the Internet.

Memory 1608 stores program instructions that are executed by, and data that are used and processed by, CPU 1602 to perform the functions of computer system 1600. Memory 1608 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 1608 may vary depending upon the function that computer system 1600 is programmed to perform. In the example shown in FIG. 16, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present systems and methods may include any and all such arrangements.

In the example shown in FIG. 16, memory 1608 may include MRI imaging data 1612, network identification routines 1614, risk scoring models and routines 1616, scoring accuracy routines 1618, classification models and routines 1620, combining routines 1622, generalized network model 1624, and operating system 1622. MRI imaging data 1612 may include data obtained from MRI imaging, as described above. Network identification routines 1614 may include software to identify a highest portion of novelty seeking associated multimodal brain networks based on MRI imaging data 1612, as described above. Risk scoring models and routines 1616 may include machine learning models and software routines to compute a plurality of scores indicating a risk of developing behaviors, as described above. Scoring accuracy routines 1618 may include software routines to determine how accurately the computed plurality of scores indicated the risk of developing the behaviors, as described above. Classification models and routines 1620 may include machine learning models and software to classify cohorts of persons (or individuals) using features of novelty seeking associated multimodal brain networks, as described above. Combining routines 1622 may include software routines to combine features of novelty seeking associated multimodal brain networks to form a model of a generalized dysfunctional novelty seeking associated multimodal brain network 1624, as described above. Operating system 1618 may provide overall system functionality.

As shown in FIG. 16, the present communications systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

### EXAMPLES

### Methods

### IMAGEN Participants

In the IMAGEN project, adolescents were recruited through local public schools at eight sites across Europe. The temperament and character inventory test (TCI) was used to evaluate NS. Alcohol consumption use was assessed by alcohol use disorders identification test (AUDIT) and Fagerstrom test for nicotine dependence (FTND). The subjects were screened for psychiatric disorders with development and well-being assessment questionnaire (DWBA), including risk rating scale scores of depression and psychosis. Gender differences on these clinical scores are shown in Table 1. The study was approved by the local ethics committees and adhered to the Declaration of Helsinki and the written informed consents were obtained. The IMAGEN adolescents completed a range of reward (the modified monetary incentive delay, MID), emotion (faces task) and reinforcement (the stop-signal task) related functional tasks and structural neuroimaging at the age of 14 and 19. Details of each task design, imaging parameters, and preprocessing strategies are presented in the “Multimodal imaging parameters and preprocessing” section.

TABLE 1
Gender difference of the studied clinical measures (Male vs Female group differences).

| Subjects | NS | AUDIT | FTND | hyperactivity | depression | psychosis |
|---|---|---|---|---|---|---|
| 14 years old | p = 0.96 | p = 0.09 | p = 0.54 | p = 0.14 | p = 0.91 | p = 0.28 |
| 19 years old | p = 0.82 | p = 0.97 | p = 0.44 | p = 0.35 | p = 1.9e−06 | p = 0.18 |
| Unseen 19 | p = 3.3e−04 | p = 3.4e−12 | p = 0.004 | p = 6.6e−06 | p = 6.6e−19 | p = 0.11 |
| years old | | | | | | |
| Patients | NA | p = 0.89 | p = 0.57 | p = 5.3e−06 | p = 0.51 | p = 0.72 |

### Multimodal Imaging Parameters and Preprocessing

### Reward Task

The participants performed a modified version of a monetary incentive delay (MID) task to test their reward processing. In short, each trial involved an anticipation phase, a response phase, a feedback phase, and a fixation period. During the anticipation phase, cues signaling the amount of reward that could be won in a given trial (large, small, or none) were shown for 4 seconds. The subject could win large or small numbers of points (10 or 2) by responding as quickly as possible to a response cue. The points were converted to food snacks (small chocolate candies) following testing (5 points per candy). The subject completed 22 trials per condition, yielding 66 trials in total. One cue (circle with two lines) signaled that a large reward could be won, another (circle with one line) signaled that a small reward could be won, and a third cue (triangle) signaled that no reward could be won in the respective trial. Following a random time interval, a response cue was displayed, and the subject was instructed to respond as quickly as possible to this cue by means of a button press. The time window in which responses were counted as “wins” was adjusted dynamically during the course of the experiment according to the subject's performance, such that on average the subject won in 66% of all trials. The response and feedback phases had a total duration of 2 seconds. Four seconds of inter-trial fixation separated the trials.

For the MID task, 40 slices were acquired in descending order (2.4 mm, 1 mm gap) using a gradient-echo T2*-weighted sequence (EPI) with the following image parameters: TR=2200 ms, TE=30 ms, and an in-plane matrix size of 64×64 pixels. A plane of acquisition tilted to the anterior-posterior commissure line (rostral>caudal) was used.

All preprocessing procedures were conducted by using SPM12 (Wellcome Trust Centre for Neuroimaging, London) and comprised slice-timing correction, spatial realignment to the middle volume, and nonlinear warping of each EPI to an EPI template. A first-level model was constructed on the unsmoothed single-subject data by using the following regressors: 1) anticipation of large reward, 2) anticipation of small reward, 3) anticipation of no reward, 4) feedback indicating large reward, 5) feedback indicating small reward, 6) feedback indicating no reward. Each regressor was included twice, once for successful trials, i.e., hits, and once for unsuccessful trials, i.e., misses. Thus, there were a total of 12 regressors. Trials in which subjects failed to respond were modeled as separate error trials. Movement parameters (3 translations and 3 rotations) from the realignment procedure were included as covariates in the first-level model for each subject. Next, contrast images of the parameter estimates were created for each subject. In the present investigation, the analysis was focused on the reward anticipation phase, i.e., greater neural responses during anticipation of Large Win than during anticipation of Small Win. Images were then smoothed with a Gaussian kernel of 5-mm full-width at half-maximum.

### Emotion Task

As stimuli, a sequence of video clips of faces was chosen that the volunteers were requested to view passively. They consisted of short (2-5s) black-and-white video clips showing faces that always started from a neutral expression, and then either turned angry or displayed a neutral movement without a particular emotional content (for example, twitching the nose). These stimuli were arranged in 18s blocks, each block including 4-7 video clips depicting faces of the same emotion (either angry or neutral). Altogether, there were five blocks of neutral faces and five blocks containing angry faces. In between two blocks of face clips, an 18s non-biological control video clip was presented. The control stimuli consisted of expanding and contracting black-and-white concentric circles of various contrasts, roughly matching the contrast and motion characteristics of the faces clips. Prior to functional MR scanning, participants were instructed that they would be presented with short video clips showing faces with angry and neutral expressions as well as moving circles. They were asked to watch the video clips carefully and lie as still as possible during the task. The same instruction was given to them directly before the task started.

For faces task, the design matrix defines the timing of Faces blocks and Control blocks, and also includes estimated motion parameters. Contrast images were calculated from angry faces minus nonbiological motion.

### Inhibition Task

Impulse control is often measured using the stop-signal task (SST) which measures brain response during successful and failed inhibition. Participants responded to regularly presented visual go stimuli (arrows pointing left or right) and were instructed to withhold their response when the go stimulus was followed (unpredictably) by a stop signal (arrow pointing upward). Stopping difficulty was manipulated across trials by varying the delay between the onset of the go arrow and the stop arrow (stop-signal delay) using a previously described tracking algorithm.

For stop-signal task, participant's design matrix included regressors for stop success trials, stop failure trials, trials on which the go response was too late, trials on which the go response was wrong (if any) and motion parameters. Contrast images were calculated for unsuccessful inhibition (stop failure) vs. an implicit baseline. The time required to stop a response, the stop-signal reaction time (SSRT), is extensively used as a clinical index of impulse control. In particular, participants with ADHD have longer SSRTs, as do chronic users of cocaine and individuals with alcohol dependence.

sMRI

High-resolution anatomical MR images were acquired, including a three-dimensional T1-weighted magnetization prepared gradient echo sequence (MPRAGE) based on the Alzheimer's disease Neuroimaging Initiative protocol. The structural image comprised 160 slices (1.1-mm thickness, TR=2,300 msec, TE=2.8 msec) and required 9 minutes for acquisition.

The sMRI data was normalized to Montreal Neurological Institute (MNI) space (a well-known template used in neuroimaging) using the unified segmentation method in SPM12, resliced to 3×3×3 mm, and segmented into gray matter (GM), white matter, and cerebral spinal fluid (CSF). Then, the GM images were smoothed with an FWHM of 8 mm Gaussian filter. Subject outlier detection was further performed using a spatial Pearson correlation with the template image to ensure that all subjects were properly segmented.

### Motion and Covariates Regression

To control confounding effects of motion artifact, multiple strategies were conducted. Subjects who have framewise displacements (FD, mean framewise displacements, mean of root of mean square frame-to-frame head motions assuming 50 mm head radius) exceeding 1 mm, and head motion exceeding 2.5 mm of maximal translation (in any direction of x, y or z) or 2.5° of maximal rotation throughout the course of scanning were removed. Six head motion parameters (3 translations and 3 rotations) in MID, faces, stop signal tasks were regressed out during task fMRI preprocessing. Correlation analysis was also performed between mean FD and the referred NS scores for each task modality. Results show that there is no significant (p=0.8,p=0.9,p=0.9 for MID, faces and stop signal task respectively) correlation between mean FD and task fMRI. In addition, the site, gender, age and handiness (total GM volume for GM) were regressed out for all the four modalities before fusion analysis.

### Non-IMAGEN Cohorts

SZ (n=147) were aggregated from fBIRN that had no current or past history of other psychiatric or neurological illness. MDD (n=81) were recruited from the West China Hospital of Sichuan. ADHD (n=320) data (http://fcon_1000.projects.nitrc.org/indi/adhd200/index.html) were obtained from the ADHD-200 project. Alcohol and drinking subjects come from Erlangen, Germany. Diagnosis of SZ, MDD and ADHD were based on Structured Clinical Interview for DSM-IV. Demographic information of each diagnosed group and adolescents can be found in Table 2.

TABLE 2
Demographic information of subjects studied

| | | | NS |
|---|---|---|---|
| | Age | Gender | (mean ± sd) |
| 14-year-olds | 14.4 ± 0.4 | 100M/139F | 125.7 ± 4.9 |
| (n = 239, selected from 1378) | | | |
| 19-year-olds | 19.0 ± 0.8 | 100M/139F | 114.7 ± 10.3 |
| (n = 239) | | | |
| Unseen 19-year-olds | 19.1 ± 0.8 | 568M/566F | 106.6 ± 10.1 |
| (n = 1134) | | | |
| Alcohol drinkers (n = 313) | 32.0 ± 9.8 | 219M/94F | NA |
| Smokers (n = 104) | 26.4 ± 4.6 | 79M/25F | NA |
| ADHD (n = 320) | 11.1 ± 2.5 | 238M/82F | NA |
| MDD (n = 81) | 29.1 ± 9.8 | 28M/53F | NA |
| SZ (n = 147) | 42.8 ± 11.8 | 108M/39F | NA |
| HC (n = 1094) | 19.9 ± 13.0 | 591M/503F | NA |

Multimodal Fusion with High-NS Scores

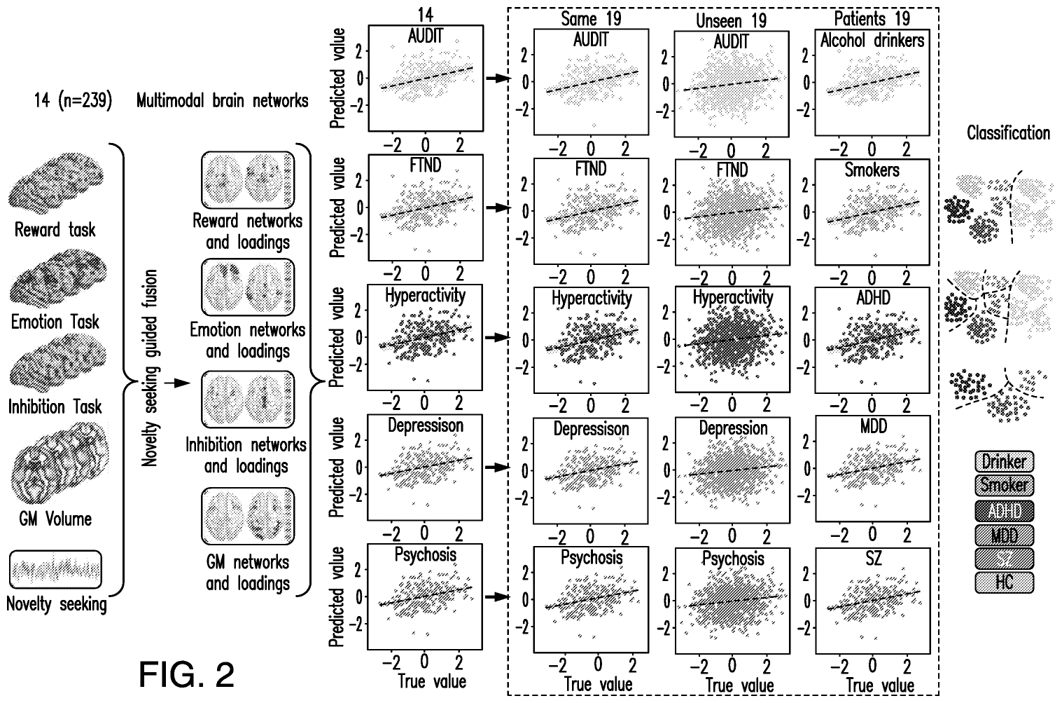

In FIG. 2, an analysis flowchart is presented where novelty seeking (NS) scores were used as a reference to guide a 4-way multimodal fusion to identify a set of multimodal imaging biomarkers, each of which was separated as positive and negative brain regions based on the Z-scored brain maps, plus the corresponding biomarker loadings, resulting in 12 features for the following prediction analysis. Multiple linear regression models were constructed for each of the five risk scores including alcohol use, smoking, hyperactivity, depression and psychosis in 14-year-olds subset. Then the same prediction models were applied to longitudinally predict each of the five risk scores of the same subjects and the large unseen healthy adolescents at the age of 19. The same models were also used to predict corresponding symptom scores of five types of patients, including alcohol drinkers, smokers, ADHDs, MDDs and SZs. Finally, binary-class and multi-class classification analysis were performed to verify the classification ability of the identified high-NS associated multimodal imaging features.

As displayed in FIG. 2, the aims of the study are achieved by using a dedicated high-NS-guided multimodal fusion, multiple cross-prediction, and multi-group classification analyses. First, a 14-year-olds adolescent subset with top 20% ranked high-NS scores (correlation between NS and other clinical measures can be found in Table 3) were selected (125.7±4.9, n=239, 100 male) as a discovery cohort to study a homogeneity subset that have the highest NS scores. These subset adolescents are assumed having higher risk to develop addiction or other psychotic disorders. Then NS scores were used as reference to guide a four-way MRI fusion on these top 20% ranked high-NS adolescents, including gray matter volume (GMV) and three task-related fMRI contrasts, to identify the high-NS associated brain regions at the age of 14. MCCAR+jICA imposes an additional constraint to maximize the column-wise correlations between loading parameters (Aₖ) and the reference NS scores (equation 1). Therefore, fusion with NS scores enables identification of a multimodal component(s) that has robust correlations with NS, which however cannot be detected by a blind multimodal fusion approach.

$${\sum }_{k , j = 1}^{2} \{ {‖ corr ( {A}_{k} , {A}_{j} ) ‖}_{2}^{2} + 2 λ \cdot {‖ corr ( {A}_{k} , ref ) ‖}_{2}^{2} \} \qquad ( 1 )$$

TABLE 3
Correlation between NS scores with other clinical measures.

| Correlation | AUDIT | FTND | hyperactivity | depression | psychosis | gender | age | handiness |
|---|---|---|---|---|---|---|---|---|
| NS | p = 0.05 | p = 0.8 | p = 0.2 | p = 0.03 | p = 0.4 | p = 0.96 | p = 0.6 | p = 0.3 |

After identifying the high-NS associated multimodal components in the high-risk subset, each component was separated as positive and negative brain regions based on the Z-scored brain maps. Thus, separate positive and negative brain masks for each of the 4 modalities (8 brain imaging networks) were obtained. The mean of the voxels within the masked region was calculated for each subject for each of the 8 networks, generating a N_{subj}×8 feature vector (Reward_P, Reward_N, Emotion_P, Emotion_N, Inhibition_P, Inhibition_N, GM_P and GM_N). Apart from the above brain imaging features, the corresponding biomarker loadings (contribution weight across subjects) of each target component (generating 4 loading features, i.e., Reward_L, Emotion_L, Inhibition_L, and GM_L) were also included. Thus, a feature matrix was formed of NS associated multimodal brain features in dimension of N_{subj}×12 in total for the prediction analysis.

As for the patient cohorts, there are no task-related fMRI data available, but only resting-state fMRI. Thus, the brain networks identified from IMAGEN tasks were used as ROIs to extract features from patients' resting state fMRI. The mean of the voxels within the ROI was calculated for each subject, generating a N_{subj}×8 feature vector for patient cohorts. The linear back-reconstruction was performed from the IMAGEN to non-IMAGEN groups to get loading features (N_{subj}×4) for each patient group. Details can be found in the “Linear back reconstruction” section.

Then the identified multimodal features (N_{subj}×12) in 14-year-olds were used to longitudinally predict five disease risks/symptoms of the same (n=239) and the unseen subjects (n≈1100, within IMAGEN) in 19-year-olds spanning drinking, smoking, hyperactivity, depression and psychosis, and clinical symptoms in five independent patient cohorts, including alcohol drinkers (n=313), smokers (n=104), ADHDs (n=320), MDDs (n=81), and SZs (n=147). Multiple linear regression models (equation (2)) were constructed for each of the five symptom/risk rating scale scores in 14-year-olds subset, separately. The predictions of symptom scores in 19-year-olds and patient groups were done by using the same regression weights obtained from 14-year-olds. Thus, no training/additional feature selection was performed on the sample of 19-year-olds and patients. Details can be found in the “Prediction” section.

$$Predicted scores = {β}_{0} + {Reward}_{P} \times {β}_{1} + {Reward}_{N} \times {β}_{2} + {Reward}_{L} \times {β}_{3} + {Emotion}_{P} \times {β}_{4} + {Emotion}_{N} \times {β}_{5} + {Emotion}_{L} \times {β}_{6} + {Inhibition}_{P} \times {β}_{7} + {Inhibition}_{N} \times {β}_{8} + {Inhibition}_{L} \times {β}_{9} + GM\_P \times {β}_{10} + GM\_N \times {β}_{11} + GM\_L \times {β}_{12} \qquad ( 2 )$$

The classification ability of the identified high-NS associated multimodal imaging features were also tested by both binary-class and multi-class classification on five patient cohorts. A linear Support Vector Machine (SVM) was used as the classification model for all the classification analyses, in which an unbiased 10-fold cross-validation framework was applied. Details can be found in the “Classification” section.

### Linear Back Reconstruction

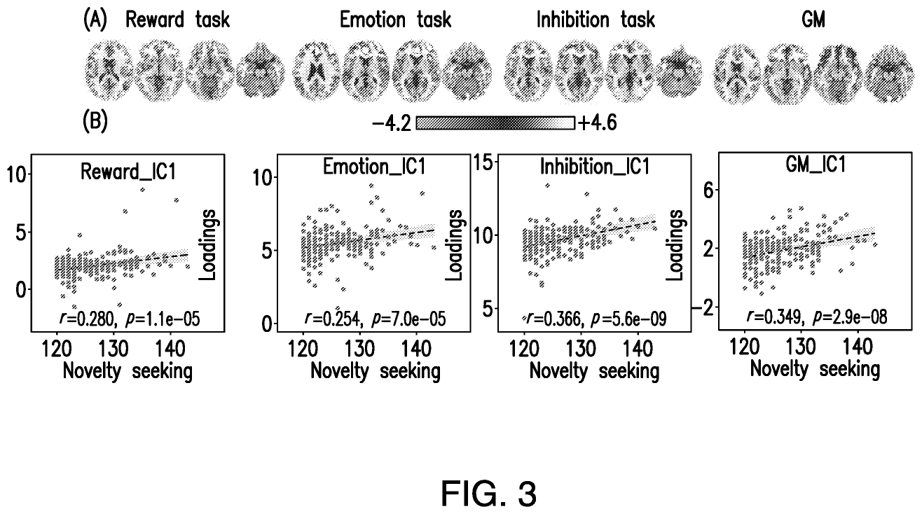

As for loading features for patient cohorts (non-IMAGEN), the linear back-reconstruction was performed from the IMAGEN cohort components to get the spatial brain maps for the patient group (FIG. 3A visualized at |Z|>2) based on a linear projection model as in Eq. (3).

$${X}_{IMAGEN , k} = {A}_{IMAGEN , k} \times {S}_{IMAGEN , k} {A}_{Patient , k} = {X}_{Patient , k} \times {( {S}_{IMAGEN , k} )}^{- 1} k = 1 , 2 , 3 , 4 \qquad ( 3 )$$

where S_{IMAGEN,k} and A_{IMAGEN,k} denote the brain components and the corresponding mixing matrix derived by MCCAR+JICA for IMAGEN cohort. X_{Patient,k} represents the preprocessed imaging feature matrixes for drinkers (X_{Drinkers,k}), smokers (X_{Smokers,k}), ADHD (X_{ADHD,k}), MDD (X_{MDD,k}) and SZ (X_{SZ,k}) patient respectively. k represents the modality (1-reward, 2-emotion, 3-inhibition, 4-GM). Consequently, the spatial maps of IMAGEN (S_{IMAGEN,k}) were used to estimate the mixing matrix of patient group (S_{Drinkers,k}, S_{Smokers,k}, S_{ADHD,k}, S_{MDD,k}, S_{SZ,k}) base on Eq. (3).

### Prediction

After multimodal feature extraction, each of the 12 vectors was normalized to mean=0, std=1 across subjects. These vectors were then treated as the linear regressors and the corresponding symptom scores were treated as the targeted measures; together, they were put into the multiple linear regression model to obtain a linear equation for an estimation of the target measures. The same regression models (beta weights) trained on the 14 years old subset over alcohol use, smoking, hyperactivity, depression and psychosis were then applied to the regressors obtained from the 19 years old (including both the same subjects and the unseen big datasets) and patient cohorts, separately. Note that different predictive models were specifically trained to predict clinical symptoms/behaviors relevant to different clinical conditions. Pearson correlations between the true and predicted values were assessed. Based on the regression models trained on the subset at 14 years old, if the estimated scores of 19 years old and independent patients' cohorts are significantly correlated with the true scores, the generalization of predictability of symptom scores based on the 12 selected regressors can be recognized to some degree.

### Permutation Test

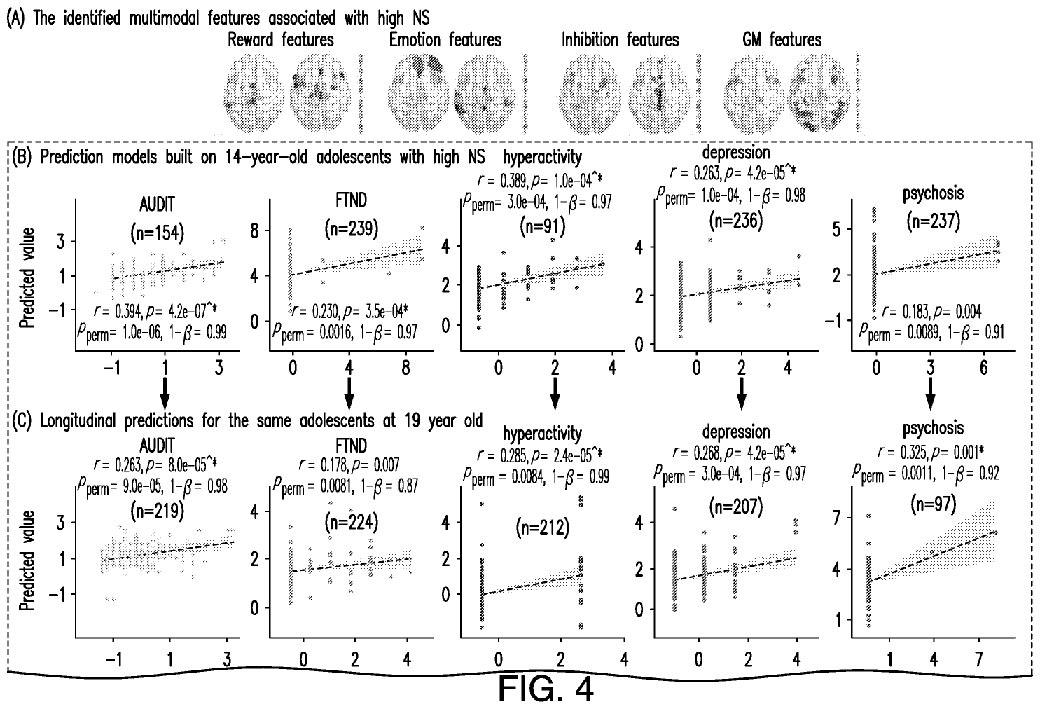

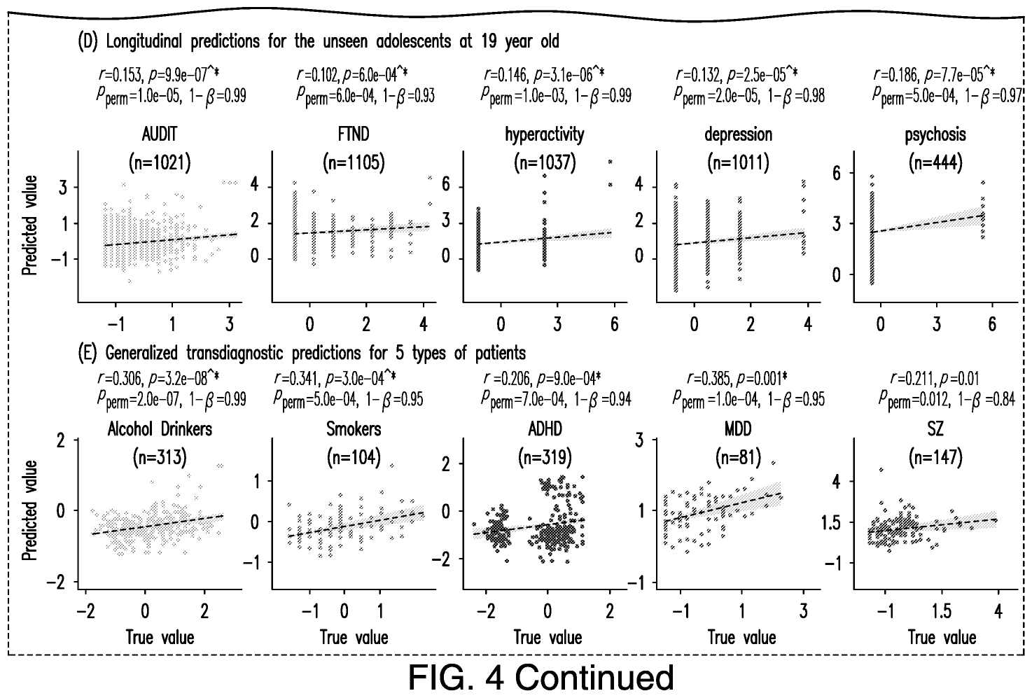

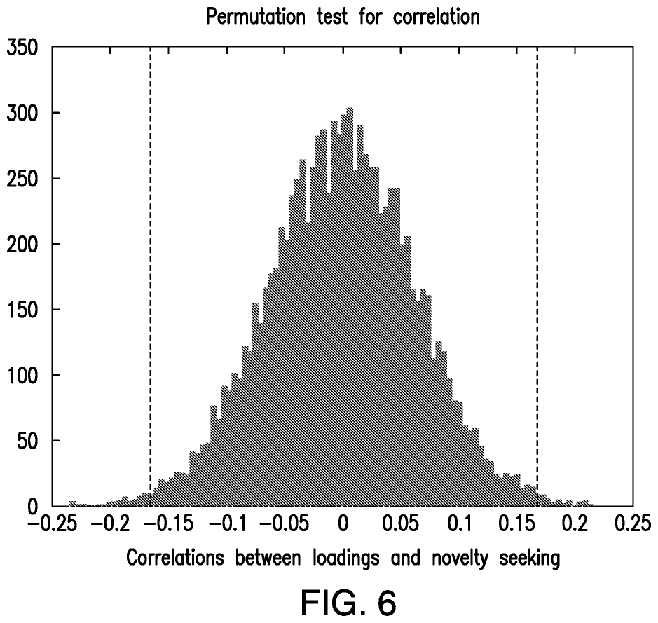

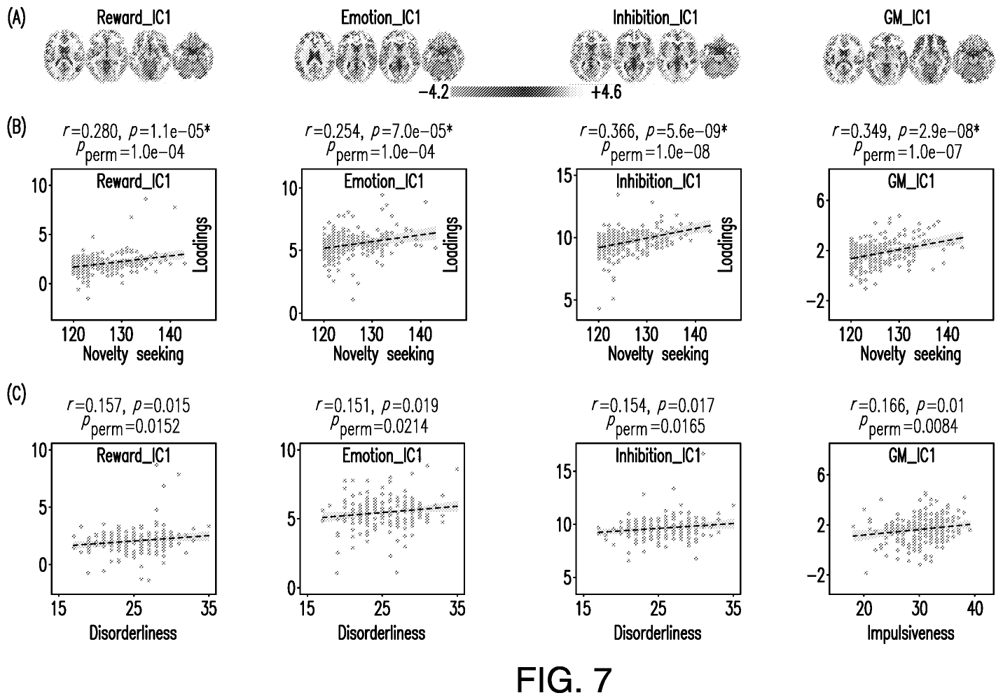

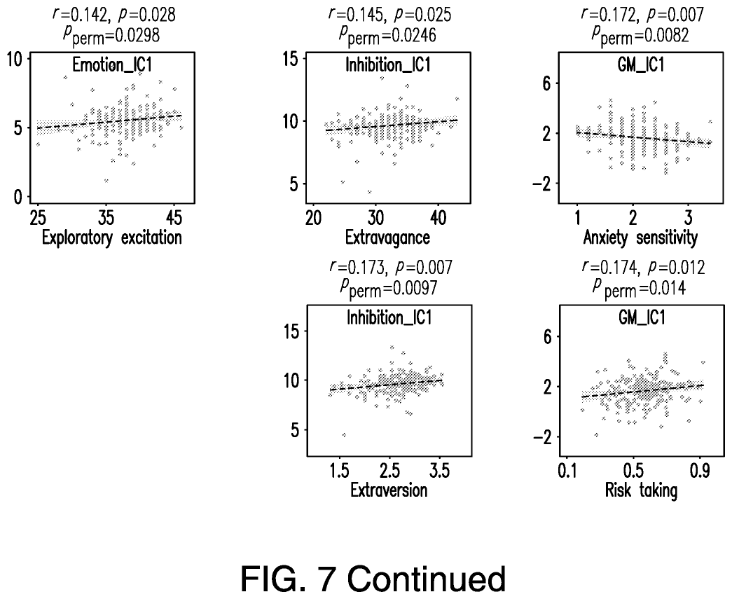

A standard permutation test for the correlations listed in the Results was also performed. This permutation test was done by randomly shuffling Y (specific personality scores, FIG. 7C) across participants and re-running the correlation analysis (between X [loadings of IC_{ref}] and Y) 10000 times in order to obtain an empirical null distribution. The number of times a correlation coefficient between X and Y exceeds the obtained sample correlation was also recorded. (r=0.166, here the correlation between GM_IC1 component and impulsiveness was taken as an example). Significance cutoffs were determined using the above permutation test (10000 permutations). As shown in FIG. 7C, the observed correlation between GM_IC1 and impulsiveness scores obtained on the original data was 0.166, while the sampling distribution of r under randomization is symmetric around 0.0 (FIG. 6), and 84 of the 10000 randomizations exceeded ±0.166 (black dotted line). This analysis quantifies the probability p=0.0084 of obtaining a particular r=0.166 between loadings of GM_IC1 and impulsiveness scores by chance. Based on the above permutation procedure, all the correlations for FIG. 3-4 were tested, where FIGS. 3A-3B show the identified high-NS associated multimodal components in 14-year-olds subset and FIGS. 4A-4E show the prediction results based on the identified high-NS associated multimodal brain imaging networks.

### Null Pattern

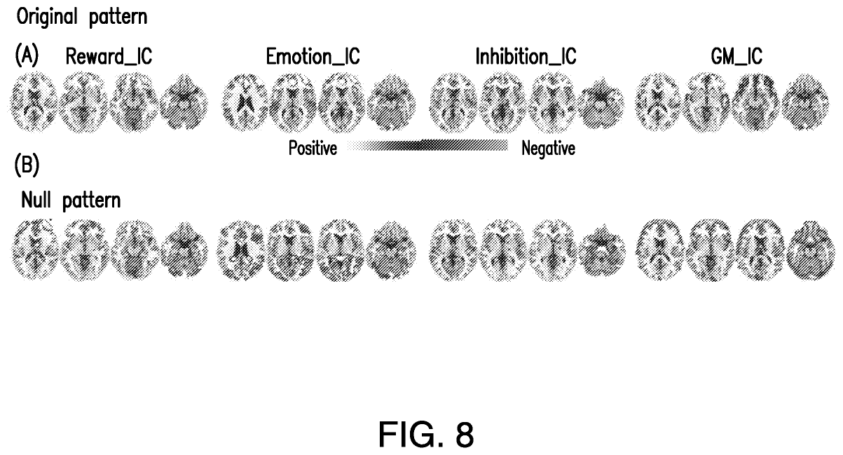

In order to see the null pattern, the reference vector (NS scores) was permuted in the supervised fusion analysis. The goal is to compute the null model of spatial patterns that are observed by chance. To do this calculation, imaging variables (e.g. [X₁, X₂, X₃, X₄]) were held constant, and the NS scores were permuted against them. Thus, each Xᵢ is randomly paired with a NS. This permuted reference was then used as reference in a supervised fusion analysis (MCCAR+jICA). By repeating this process, a large number of times (1000), 1000 four-MRI covarying patterns associated with the permuted NS scores were obtained. The number of times each spatial pattern occurs was also recorded. Here, the most frequently occurring voxels (those which occur more than 70% of the time) associated with the permuted NS scores were presented, as shown in FIG. 8. Note that the permuted null pattern of spatial maps is different from the comprised high-NS associated network, confirming that the observed pattern as presented in these results are specific to the NS measures but not a random null pattern.

### Contribution Weights of Different Modalities

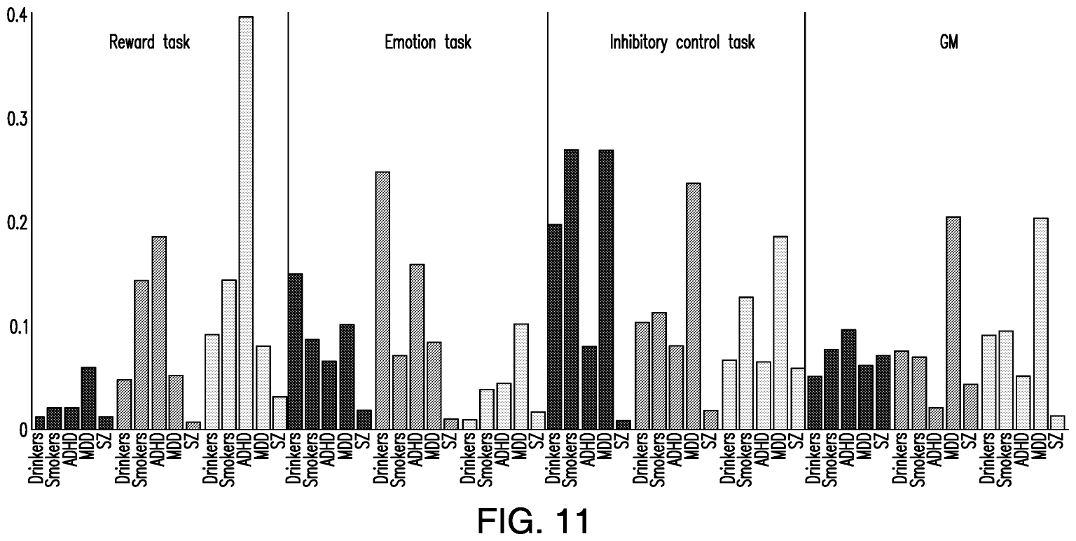

Although the identified high-NS associated brain ROIs can be used to predict 5 different types of symptom scores, the contribution weights of imaging features for each disease model are different. FIG. 11 displayed the absolute beta weights (β₁, β₂, . . . , β₁₂ as in Eq (1)) of the multivariate linear regression models for 4 modalities: reward task, emotion task, inhibition task and GM, in which dark gray, medium gray, and light gray bars represent positive map, negative map and loadings respectively. The positive (Z>0) and negative (Z<0) brain networks are extracted based on the Z-scored brain maps. When comparing across different modalities, it is evident that the task-related fMRI data contributes more than GM in the five-symptom prediction. When comparing within 3 fMRI tasks, the inhibitory control task contributes more to the prediction than the reward and emotion tasks. Regarding the specificity of different brain networks to different brain disorders, it was found that the positive brain networks (FIG. 3A) of inhibition control task (including prefrontal, insular and thalamus) are more relevant to drinking, smoking and depression. The negative brain networks of emotion task (including thalamus, amygdala and temporal cortex) are more relevant to drinking and ADHD. This means that functional synchronized inhibition network in prefrontal, insular and thalamus would represent as an early risk indicator for developing drinking and smoking, while the dysfunction of facial emotion-related regions in thalamus, insular and middle temporal cortex would suggest higher risk for developing ADHD in future.

### Classification

The classification ability of the identified high-NS associated multimodal imaging features were also tested by both binary-class and multi-class classification on five patient cohorts. Linear SVM implemented in the Classification Learner app (MATLAB2019) was used as the classification model for all the classification analyses. Note that there are imbalanced subject number in each group, with MDD having the fewest (n=81). Re-sampling was done for large sample size groups in order to get a comparable sample size for each group. Here, take classifying ADHD, MDD and SZ as an example. Undersampling by randomly removing some subjects from ADHD and SZ groups was performed to get comparable subject numbers (n=100) with the MDD group. The above random re-sampling was repeated 200 times. Within each re-sampling, an unbiased 10-fold cross-validation framework, in which nine of the ten folds were used as the training data and the remaining fold was used as the testing data, was applied. The predictability of the identified high-NS associated multimodal features in differentiating between all patients and controls (2-class), as well as 6-class, 5-class and 3-class classifications were tested.

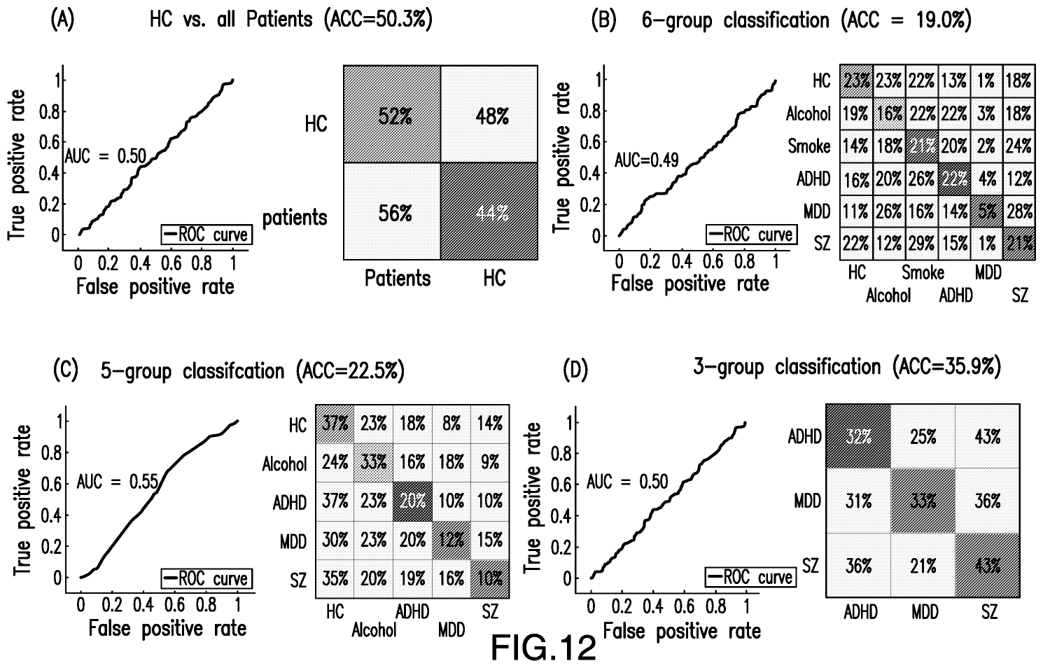

A null distribution in classification by permuting the class labels 1000 times in the classification analysis was also provided. Results (FIG. 12) show that the null distribution accuracies are approximated as 1/C<50% (C represent group numbers) as a random distributed accuracy. This means that can reject the null distribution for the current classification result.

### Example 1: High-NS Associated Multimodal Brain Networks at Age 14

As shown in FIG. 3A, spatial brain maps were visualized at |Z|>2 and correlated to scatter plots between NS scores and loadings of component for each modality. One linked reward-emotion-inhibition-GM component was identified by supervised fusion, showing significant correlations between its loadings and the high-NS scores in all modalities (r=0.280*, pₚₑᵣₘ=1.0e-04; r=0.254*, pₚₑᵣₘ=1.0e-04; r=0.366*, pₚₑᵣₘ=1.0e-08; r=0.349*, pₚₑᵣₘ=1.0e-07 for reward, emotion, inhibition tasks and GM, respectively). pₚₑᵣₘ represents the p values from the permutation test (FIG. 6), with details in the “Permutation test” section. As shown in FIG. 7, this component is also correlated between NS scores and loadings of component for each modality (FIG. 7B) and other personality scores and loadings for each modality (FIG. 7C). The identified brain regions are summarized in Table 4 for task components and GM (Talairach labels), respectively. In order to confirm that the extracted high-NS associated multimodal patterns are specific to the NS measures but not a random pattern, the NS scores were permuted in the supervised fusion analysis. In FIG. 8 shows the covarying pattern for the original cognitive scores (FIG. 8A) and the most frequently occurring (voxels with more than 60% occurrences) covarying pattern associated with 1000 times permuted cognitive scores (FIG. 8B). Note that the random pattern (FIG. 8) is very different from the identified high-NS associated network, supporting the specificity of the relationship to NS (FIG. 3A).

TABLE 4
Anatomical information of the identified high-NS associated components in 14 years old subset.

| | | |
|---|---|---|
| Brodmann Area | volume (cc) | random effects: Max Value (x, y, z) |

Positive

| | | | |
|---|---|---|---|
| Reward_IC1 Area | | | |
| Parahippocampal Gyrus | 19, 27, 30, 35, 36 | 4.0/1.5 | 5.2 (−9, −38, 5)/NaN |
| Thalamus | | 1.0/0.9 | 2.5 (−3, −14, 3)/2.1 (9, −17, 15) |
| Superior/Middle Temporal | 19, 21, 22, 38, 39 | 1.0/1.7 | 2.6 (−45, −18, −4)/2.4 (50, −1, −10) |
| Gyrus | | | |

Negative

| | | | |
|---|---|---|---|
| Anterior Cingulate | 24, 25, 31, 32, 33 | 4.8/2.8 | 4.9 (−6, −2, 28)/4.3 (6, 4, 27) |
| Caudate | | 0.6/1.2 | 3.2 (−9, 7, 16)/3.9 (6, 12, 13) |
| Parahippocampal Gyrus | 28, 34, 35 | 0.3/1.8 | NaN/3.6 (33, −7, −17) |
| Insula | 13 | 3.3/1.6 | 2.9 (−33, −2, 17)/3.1 (33, 18, 13) |
| Middle/Inferior Frontal | 6, 8, 9, 10, 11, 13, | 5.2/1.6 | 2.7 (−42, 4, 30)/2.1 (33, 35, −7) |
| Gyrus | 44, 45, 46, 47 | | |

Positive

| | | | |
|---|---|---|---|
| Faces_IC1 Area | | | |
| Anterior Cingulate | 10, 24, 25, 32, 33 | 5.4/6.2 | 4.4 (0, 39, 20)/5.6 (3, 41, 6) |
| Superior/Middle/Inferior | 6, 8, 9, 10, 11, 13, | 7.2/20.5 | 3.1 (−30, 38, −4)/4.8 (30, 62, 11) |
| Frontal Gyrus | 32, 45, 46, 47 | | |
| Insula | 13, 47 | 1.5/4.1 | 2.0 (−39, 12, −1)/4.2 (45, 6, 2) |
| Caudate | | 0.3/0.3 | 2.3 (−6, 3, 0)/2.5 (9, 18, 7) |

Negative

| | | | |
|---|---|---|---|
| Superior/Middle Temporal | 13, 21, 22, 38, 39, | 11.0/5.7 | 4.0 (−56, −40, 16)/3.8 (48, −20, 1) |
| Gyrus | 41, 42 | | |
| Parahippocampal Gyrus | 19, 30, 35, 36, 37 | 0.8/0.8 | 2.2 (−21, −56, −5)/2.7 (24, −1, −18) |
| Thalamus | | 0.9/0.3 | 2.0 (−9, −17, 6)/1.8 (12, −14, 6) |

Positive

| | | | |
|---|---|---|---|
| Inhibition_IC1 Area | | | |
| Insula | 13 | 3.6/2.6 | 3.4 (−42, −20, 15)/2.7 (42, −17, 1) |
| Superior/Middle/Inferior | 6, 9, 13, 46, 47 | 0.8/1.9 | 2.2 (−42, 30, 15)/2.9 (33, 14, −13) |
| Frontal Gyrus | | | |
| Superior Temporal Gyrus | 13, 21, 22, 38, 41 | 1.9/2.1 | 2.8 (−39, 11, −21)/2.9 (33, 8, −21) |
| Parahippocampal Gyrus | 34, 35 | 0.4/1.1 | 2.0 (−27, −1, −23)/2.4 (30, 2, −18) |
| Anterior Cingulate | 10, 24, 25, 32 | 0.8/0.4 | 2.0 (−9, 35, 9)/3.2 (3, 10, 27) |
| Thalamus | | 0.2/0.3 | 1.7 (−6, −23, 7)/1.9 (12, −23, 7) |

Negative

| | | | |
|---|---|---|---|
| Caudate | | 1.5/1.4 | 4.9 (−6, 15, 8)/5.2 (6, 7, 13) |
| Medial Frontal Gyrus | 6, 8, 9, 10 | 1.1/1.6 | 3.0 (0, −9, 50)/2.8 (3, −9, 53) |

Positive

| | | | |
|---|---|---|---|
| GM IC1 Area | | | |
| Insula | 13 | 2.4/2.5 | 3.7 (−39, −34, 18)/2.4 (42, −28, 18) |
| Superior/Middle Temporal | 13, 21, 22, 38, 39, | 7.8/7.6 | 3.3 (−42, −31, 15)/3.5 (36, 22, −29) |
| Gyrus | 41 | | |
| Middle Frontal Gyrus | 6, 8, 9, 10, 11, | 2.4/4.3 | 2.4 (−42, 17, −13)/3.3 (42, 24, 24) |
| | 46, 47 | | |
| Parahippocampal Gyrus | 34 | 0.1/0.5 | 1.8 (−9, −44, 0)/2.1 (21, 5, −18) |

Negative

| | | | |
|---|---|---|---|
| Superior/Middle/Inferior | 6, 8, 9, 10, 13, | 2.8/3.3 | 3.0 (−33, 16, 32)/5.1 (33, 19, 32) |
| Frontal Gyrus | 45 | | |
| Superior/Middle/Inferior | 19, 20, 21, 22, | 5.8/4.7 | 4.9 (−45, −38, 7)/3.8 (45, −61, 1) |
| Temporal Gyrus | 37, 39, 41 | | |
| Middle Occipital Gyrus | 18, 19, 37 | 3.7/4.0 | 3.8 (−27, −81, 12)/3.7 (30, −75, 15) |
| Inferior Parietal Lobule | 7, 39, 40 | 2.2/4.2 | 3.2 (−33, −62, 39)/3.7 (48, −39, 27) |

### Example 2. Predictive Models Trained at Age 14

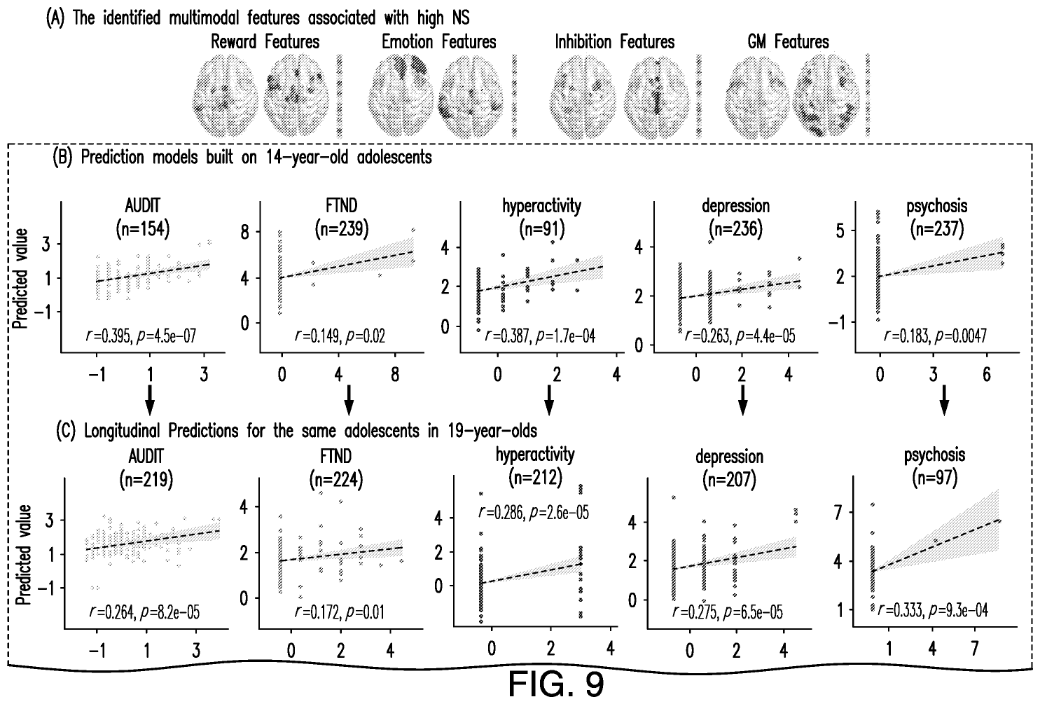

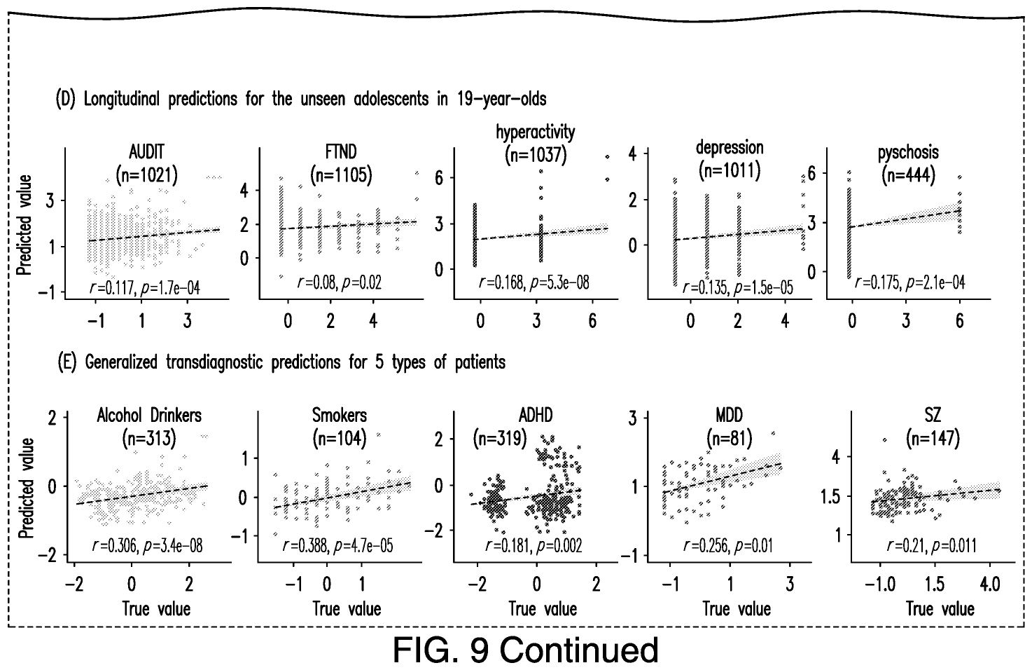

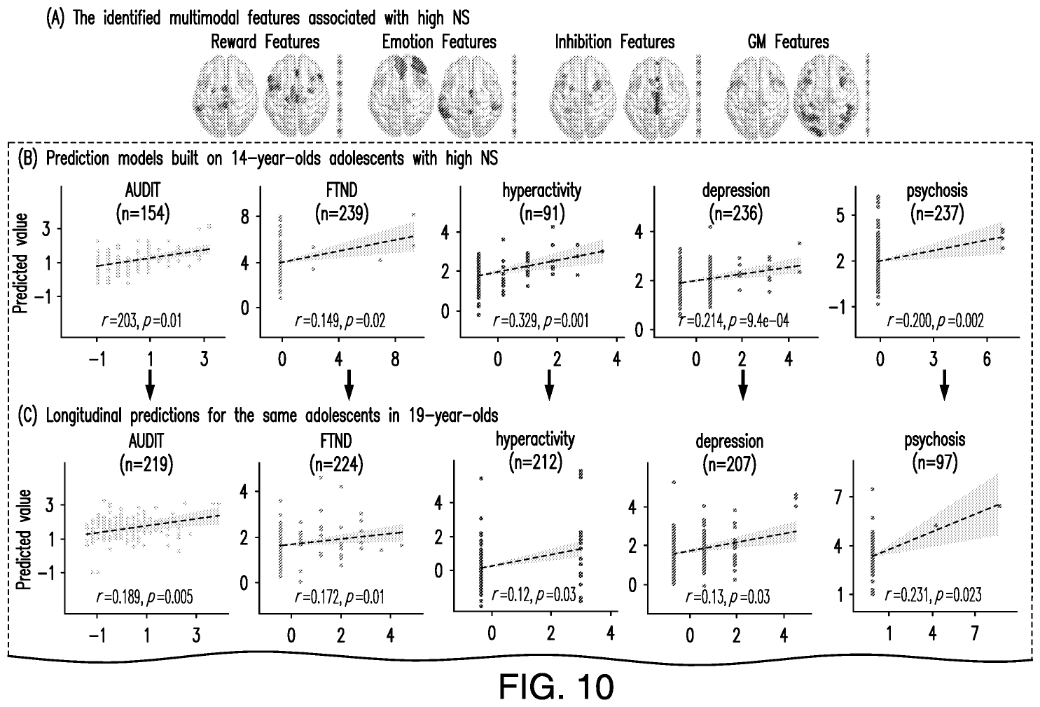

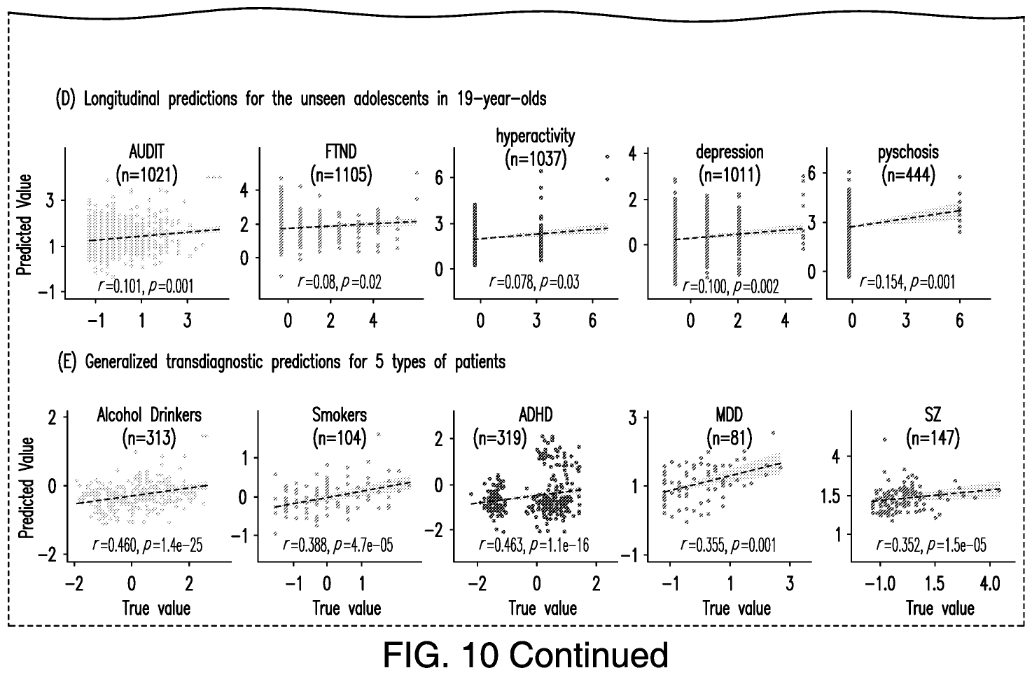

Pearson correlations of r=0.394, 0.230, 0.389, 0.263, 0.183 were achieved between the estimated symptom/risk scores and its true values for drinking, smoking, hyperactivity, depression and psychosis in the 14 years old youth subset (FIG. 4B), with the corresponding permuted p values of p_perm=1.0e-08, 0.0016, 3.0e-04, 1.0e-04, 0.0089 respectively. Details about the permutation tests can be found in Supplementary “Permutation test”. Importantly, the prediction results remain significant even regressing out gender (FIG. 4, FIG. 9). Note that some of the predicted measures are with discrete values (e.g., FIG. 4B smoker), hence, Spearman correlation was also calculated in addition to the Pearson correlation, which remain significant for the five risk score predictions as well (FIG. 10).

### Example 3: Within IMAGEN: Longitudinal Risk Prediction in 19 Year Olds

In FIG. 4A-4E, prediction results based on the identified high-NS associated multimodal brain imaging networks are presented. FIG. 4A shows positive (dark regions on left side brain image for each of the features) and negative (dark regions on right side brain image for each of the features) brain maps of the Z-scored components, and the corresponding loading parameters (columns). Loadings represent the contribution weight of the corresponding component across subjects. FIG. 4B presents regression models trained on 14-year-olds high-risk adolescent subset on five different risk scores. FIG. 4C show within-IMAGEN longitudinal predictions on five risk scores for the same 19-year-olds adolescents (n=239) using the same prediction models as in FIG. 4B. FIG. 4D shows within-IMAGEN longitudinal predictions for the other unseen 19-year-olds adolescents (n≈1100). FIG. 4E is a generalized prediction for independent patients diagnosed as drinking, smoking, ADHD, MDD or SZ (n=964). The n in each subplot represents the number of subjects with that kind of risk scores. r represents correlation between true values and the predicted values. Here pₚₑᵣₘ represents the p values of permutation test, 1−β represents the statistical power. * indicates FDR correction for multiple comparisons and \hat{ } indicates Bonferroni correction. A summarized table on the prediction accuracy estimation by including i) Pearson correlation, ii) Spearman correlation, iii) partial correlation by regressing out gender, iv) normalized root mean squared prediction error (NRMSE), v) permutation test, vi) statistic power can be found in Table 5.

The same predictive models and the brain ROIs identified on the subset in 14-year-olds were generalized to predict AUDIT (r=0.263, pₚₑᵣₘ=9.0e-05), FTND (r=0.178, pₚₑᵣₘ=0.0081), hyperactivity (r=0.285, pₚₑᵣₘ=0.0084), depression (r=0.268, pₚₑᵣₘ=3.0e-04) and psychotic (r=0.325, pₚₑᵣₘ=0.0011) rating scores on the same subjects at age 19 (FIG. 4C), when most psychopathological symptoms become manifest at this age. More importantly, the same models can also be generalized to predict risks for a large number of new, previously unseen subjects in 19-year-olds on AUDIT (n=1021, r=0.153, pₚₑᵣₘ=1.0e-05), FTND (n=1105, r=0.102, pₚₑᵣₘ=6.0e-04), hyperactivity (n=1037, r=0.144, pₚₑᵣₘ=1.0e-03), depression (n=1011, r=0.132, pₚₑᵣₘ=2.0e-05) and psychotic ratings (n=444, r=0.186, pₚₑᵣₘ=5.0e-04), as shown in FIG. 4D. Similarly, the results calculated with Spearman correlations (FIG. 10) or with gender controlled (FIG. 9) still remain significant. For psychosis, the variability of psychosis scores is not homogeneous, i.e., most of the adolescents do not have psychotic symptoms; only few of them have a risk for developing psychosis, but they are not diagnosed as schizophrenia. Thus, a two-sample T test of these outlier subjects between the other subjects was also performed. For the outliers with significantly higher true psychotic rating scales, the predicted values were also significantly higher (p<1.0e-20) than the others. Longitudinal prediction on the same subjects also hold sufficient statistical power.

### Example 4: Non-IMAGEN: External Disease Severity Predictions

Both the prediction models and the brain ROIs identified in 14-year-olds can be successfully generalized to predict symptoms for five types of diagnosed patients, spanning AUDIT for drinkers (r=0.306, pₚₑᵣₘ=2.0e-07), FTND for smokers (r=0.341, pₚₑᵣₘ=5.0e-04), inattentive for ADHD (r=0.206, pₚₑᵣₘ=7.0e-04), HDRS for MDD (r=0.385, pₚₑᵣₘ=1.0e-04) and PANSS negative for SZ (r=0.211, pₚₑᵣₘ=0.0122), as displayed in FIG. 4E. The predictions were adequately powered (1−β=0.99, 0.95, 0.94, 0.95, 0.84), and also remain significant with either spearman correlation or gender-controlled (FIG. 9-10). Although the identified high-NS associated brain ROIs can be used to predict five different types of symptom scores, the contribution weights (β₁, β₂, . . . , β₁₂ as in Eq (2)) of imaging features for each disease model are different (FIG. 11).

### Example 5: Potential to N-Way Classification

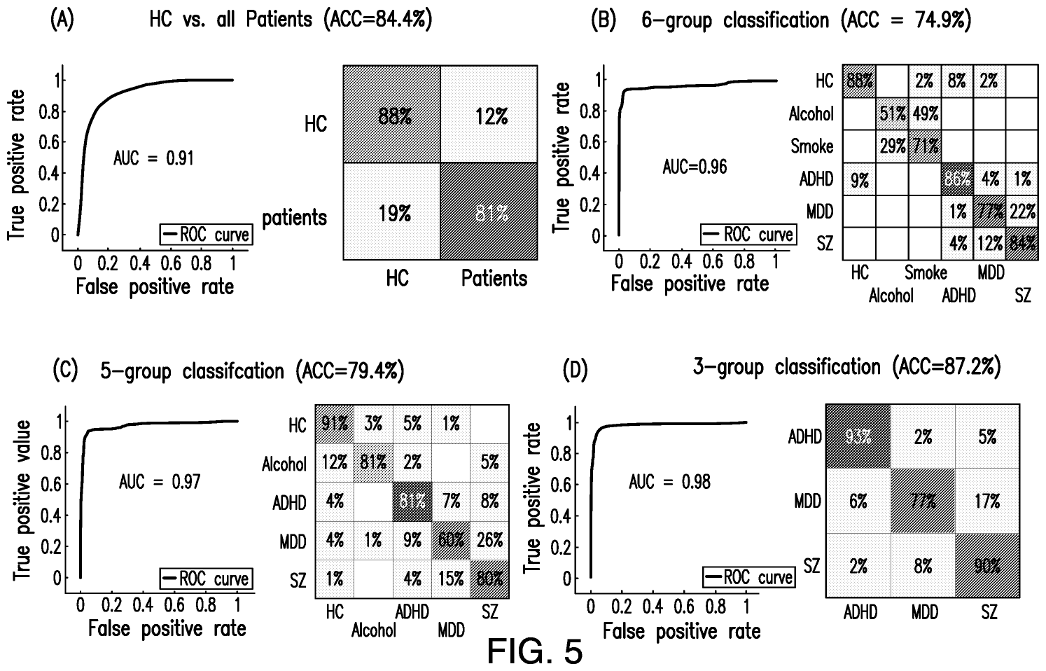

Besides predicting the continuous values such as disease severity, the potential of the identified high-NS associated multimodal features to discriminate multiple types of patients (multi-class classification) were also tested. The same imaging features as used in symptom prediction were adopted, with the site, age and gender regressed out prior to classification. As displayed in FIG. 5A, the binary classification accuracy between controls (n=1094) and all kinds of patients (n=965) is of 84.4%. For multi-group classification, accuracy of 74.9% (FIG. 5B), 79.4% (FIG. 5C) and 87.2% (FIG. 5D) were achieved respectively for 6-group classification among HCs, alcohol drinking, smoking, ADHD, MDD and SZ, 5-group classification among HC, alcohol drinking, ADHD, MDD and SZ, and 3-group classification among ADHD, MDD and SZ. The rows in each confusion matrix show the true group label, and the columns show the predicted label. The diagonal darker cells (true positive rate) show where the true labels and predicted labels match. The off-diagonal cells (lighter gray, false negative rate) represent the misclassified percentage. Particularly, the AUC is 0.98 for classifying ADHD, MDD and SZ; and in all cases, the AUC is higher than 0.91, showing promise for clinical utility of the identified NS-associated biomarkers. Furthermore, the classification results were not consistent with the null distribution (FIG. 12).

### Example 6: A Shared Multimodal Network

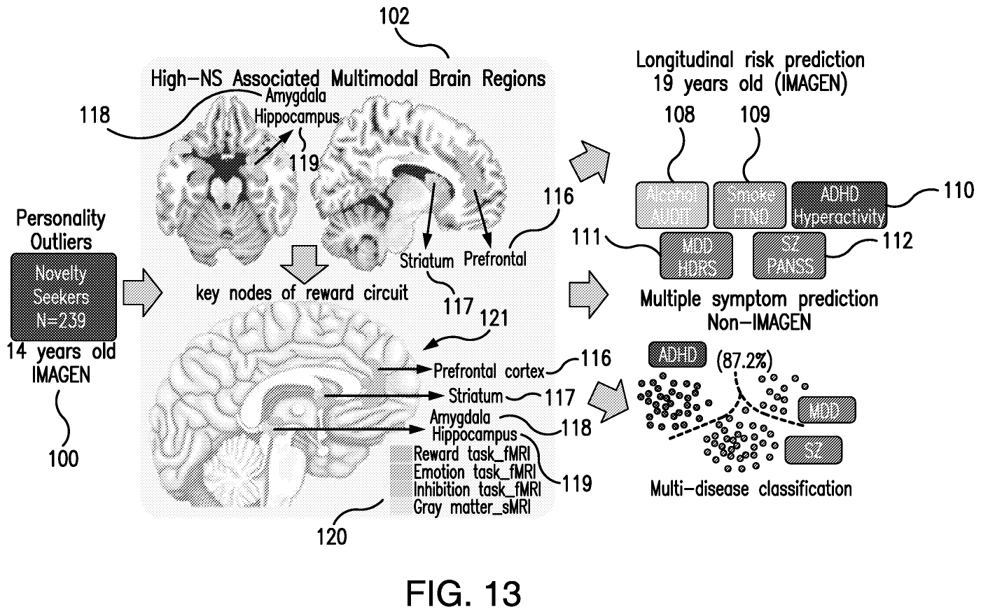

In order to find a generalized dysfunctional brain network spanning drinking, smoking, hyperactivity, depression and psychosis, all the identified high-NS-associated brain regions (102) across 4 modalities (FIG. 13) were overlaid, with different task fMRI (120) capturing different brain activated subregions (121). FIG. 13 is an illustration of the present invention showing a contribution of personality (novelty seeking), neural response (functional activation in reward, emotion and inhibition) and structural (GM volume) covariation that captures a generalized multimodal reward circuit, which may serve as a trans-diagnostic neuroimaging biomarker to predict disease risks or severity shared among five disorders. Consequently, the prefrontal cortex (116), striatum (117), amygdala (118) and hippocampus (119) are shown as the most consistent brain regions over the four modalities, which are also the key nodes of the human reward related system.

### Discussion

Based on rigorous longitudinal and transdiagnostic cross validation leveraging six independent datasets, the data-driven analysis revealed that there is a degree of dependence among drinking, smoking, ADHD, MDD and SZ and suggested that an apparent partially shared deficit in addiction and psychiatric disorders may arise from a common NS associated dysfunction. Results suggest that 1) high-NS in adolescents is associated with alterations in the reward related system that may implicate higher risk for subsequent development of the above-mentioned disorders. 2) The identified high-NS associated multimodal network may serve as a transdiagnostic neuroimaging biomarker to predict disease severity as well as classify among ADHD, MDD and SZ.

Specifically, the most spatially consistent brain regions identified associated with high-NS in four modalities were the prefrontal cortex, striatum, amygdala and hippocampus. Both ventral striatum and prefrontal cortex are key components of the reward system, while the amygdala and hippocampus are core regions involved in the regulation of reward. Moreover, it is striking that a common set of imaging signatures involving the reward related system may predict individuals on their subsequent development of the five dissimilar clinical syndromes here studied.

This work also helps re-focus the clinical community on the risk biomarker identification. That the high-NS associated multimodal reward related imaging features differentiate between patients and healthy controls with 84.4% accuracy is informative but not particularly useful. However, the 87.2% accuracy in classification amongst SZ, MDD and ADHD groups is clinically significant. Because about 50% of psychiatric patients suffer from comorbidity of at least one additional lifetime diagnosis, reliable diagnostic classification remains challenging. For example, prevalence of depression in schizophrenia is 25% and adolescent depression may progress to schizophrenia. Furthermore, the comorbidity of schizophrenia and depression makes the diagnosis of schizoaffective disorder particularly difficult, especially in adolescence. Though diagnostic classification may be straightforward in many patients, there remains a significant subgroup, often of hard to treat patients, for whom an additional biomarker analysis may increase diagnostic reliability and support treatment decisions. Hence this study's ability to discriminate between depression and schizophrenia has a useful clinical application. For these patients, performing a 10-minute sMRI and resting state fMRI acquisition is potentially feasible. Although it was emphasized that the identified high-NS associated multimodal reward circuit may serve as a common dysfunction underlying drinking, smoking, ADHD, MDD and SZ, its specificity in discriminating among ADHD, MDD and SZ indicates that this common reward network may work differently or alter to a different extent among these three disorders.

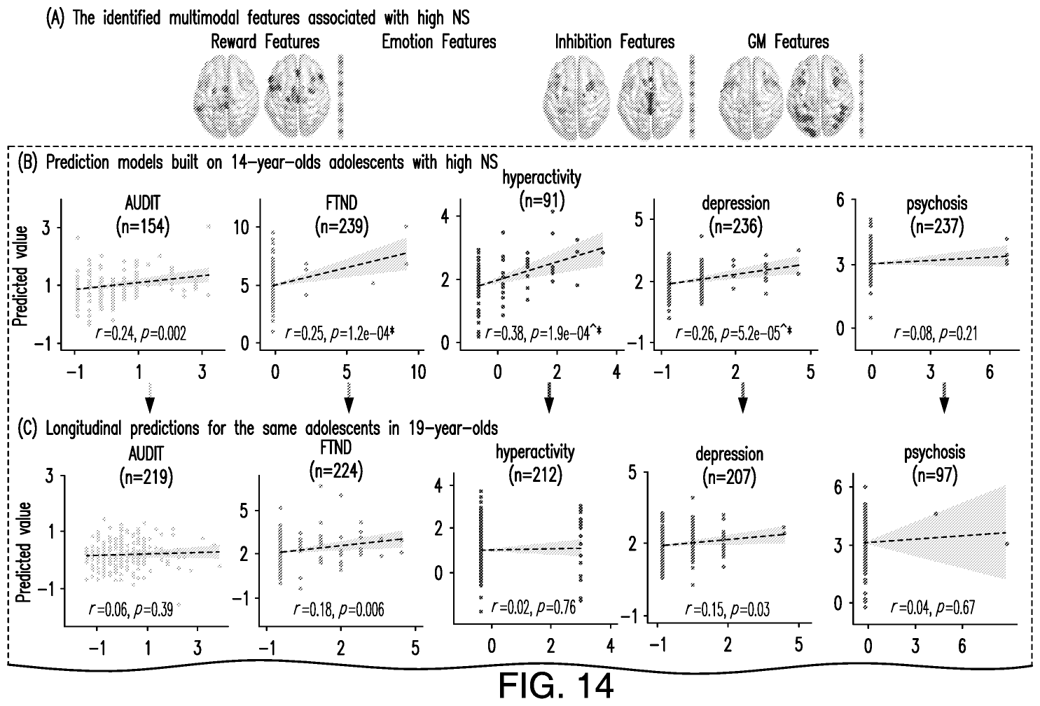

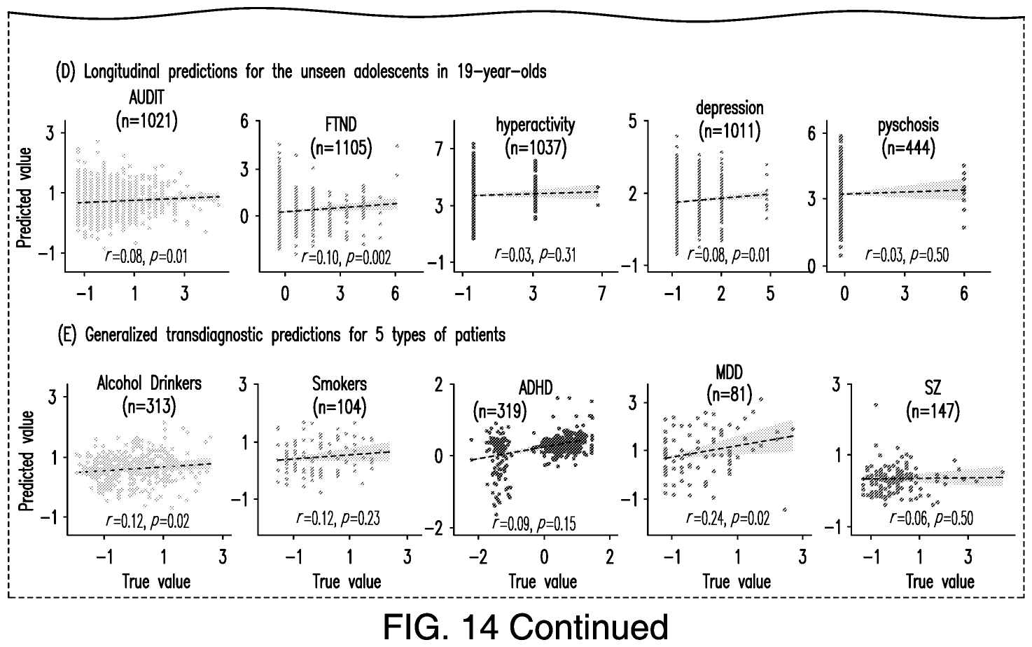

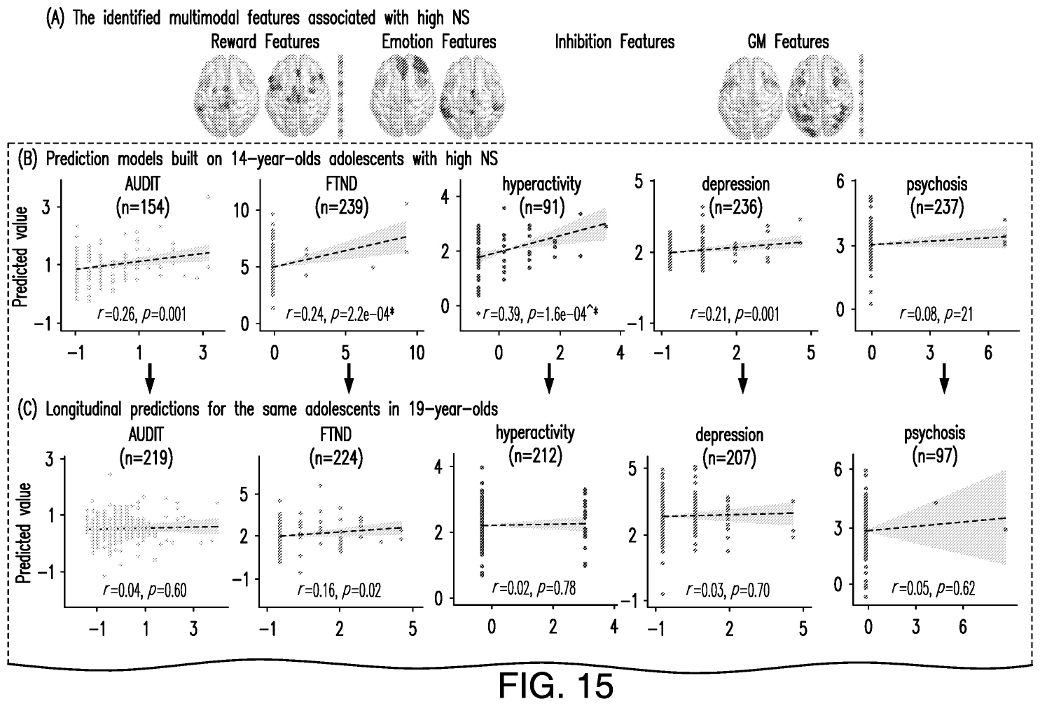

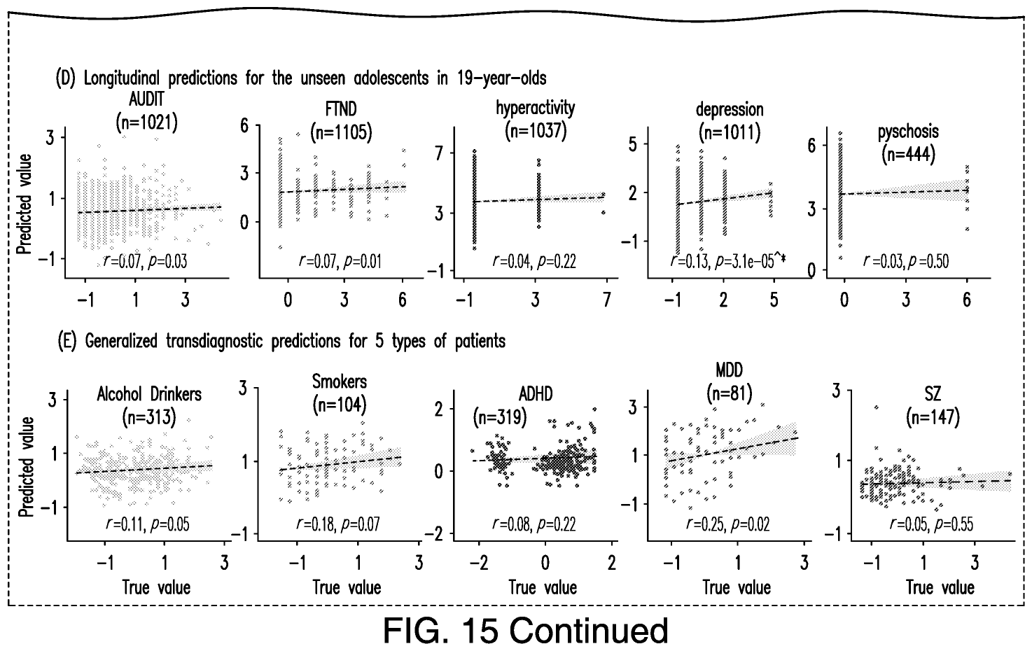

Note that for the external patient cohorts, only resting state fMRI data existed instead of the same task-related fMRI data as in IMAGEN. Still, the same ROIs from resting fMRI as those identified from task-related fMRI were extracted. The structural scans of non-IMAGEN cohorts also show certain difference from the IMAGEN/ADNI protocol. However, there is excellent reproducible performance from task to rest brain networks, without specifically matching the imaging parameters, either in prediction or in classification. This suggests that it is the combination of the subregions of the reward network from each task modality playing the most important role in prediction, which is also verified when removing one predictor related to either emotion processing or inhibition control (FIG. 14 and FIG. 15 respectively (the predictions are not significant (p>0.05) in the gray plots (* indicates FDR correction for multiple comparisons and \hat{ } indicates Bonferroni correction)); consequently, the prediction accuracy and generalizability in both longitudinal adolescents and external patient cohorts decreased.

Embodiments of the present systems and method may use other personality or social functional features to serve as a reference, such as HA, which is more associated with MDD than NS. For example, it is possible that the HA-associated multimodal features may achieve better prediction for a single disorder, such as MDD. The present invention presents systems and methods for identifying a common and generalized imaging pattern emerging in adolescence, that predicts the development of psychiatric disorders. Note that though some of the predicted measures (FIG. 4) involve discrete values, the invention also provides several different predictive accuracy estimation strategies (Table 3), and each of these criteria provides a different approach to measure the predictive accuracy. Although site, age and gender were regressed out from the multimodal feature matrix prior to classification analysis, site and age (the ADHD group is younger than the other groups) should be considered as potential confounding factors when interpreting the classification results. The study confirms that ADHD subjects had no current or past history of other psychiatric or neurological illness. However, for the other non-IMAGEN patient groups (SZ and MDD), comorbidity assessments for smoking and drinking were not available. Future analysis should examine the classification ability of the identified high-NS associated multimodal features on comorbid psychiatric conditions.

### CONCLUSION

The invention provides a specific brain network involving reward-related structures (i.e., prefrontal cortex, striatum, amygdala and hippocampus) that underlie the personality trait of novelty-seeking in mid-adolescence. Variation in this network predicts the development of various dysfunctional behaviors in late adolescence. It also predicts symptom severity in the corresponding clinical populations (i.e., smokers, alcoholics, ADHD, SZ and MDD). Finally, this network variation accurately classifies amongst the ADHD, SZ and MDD groups, highlighting the potential of a multimodal neuroimaging approach for future biomarker development. Collectively, the present invention goes beyond a specific psychiatric condition to identify shared neuroimaging patterns in multiple brain disorders by multimodal fusion, and presents the role of transdiagnostic risk factors by both longitudinal risk prediction and cross patient classification validation.

What is claimed is:

1. A method, implemented in a computer system comprising a processor, memory accessible by the processor, and computer program instructions stored in the memory and executable by the processor, the method comprising:
identifying a highest portion of novelty seeking associated multimodal brain networks in a first cohort of persons based on Magnetic Resonance Imaging data;
computing a plurality of scores, each score indicating a risk of developing one of a plurality of specified behaviors;
determining, for the first cohort of persons at a later time, how accurately the computed plurality of scores indicated the risk of developing each of the plurality of specified behaviors;
classifying at least a second person not belonging to the first cohort of persons using features of those novelty seeking associated multimodal brain networks that indicated the risk of developing the specified behaviors with greater than a predefined accuracy; and
displaying to a user the risk of the second person of developing any of the plurality of specified behaviors.

2. The method of claim 1, wherein the novelty seeking associated multimodal brain networks comprise at least one of the thalamus, the prefrontal cortex, the insular cortex, the mid temporal lobe, the striatum, the amygdala, and the hippocampus.

3. The method of claim 1, wherein the plurality of specified behaviors comprises at least some of alcohol drinking, smoking, hyperactivity, depression, and psychosis.

4. The method of claim 1, wherein the Magnetic Resonance Imaging data comprises a multiple MRI fusion, including gray matter volume (GMV) and a plurality of task-related fMRI contrasts.

5. The method of claim 4, wherein the plurality of task-related fMRI contrasts comprises at least some of a modified monetary incentive delay task, a face emotion identification task, and a stop-signal task.

6. The method of claim 1 further comprising combining the features of those novelty seeking associated multimodal brain networks that indicated the risk of developing the specified behaviors with greater than a predefined accuracy to form a model of a generalized dysfunctional novelty seeking associated multimodal brain network.

7. The method of claim 1, wherein classifying the risk of developing any of the plurality of specified behaviors comprises at least one of: a regression model, a machine learning model, an image analysis, a support vector machine, a binary classification, or a multi-class classification.

8. A system comprising a processor, memory accessible by the processor, and computer program instructions stored in the memory and executable by the processor to perform:
identifying a highest portion of novelty seeking associated multimodal brain networks in a first cohort of persons based on Magnetic Resonance Imaging data;
computing a plurality of scores, each score indicating a risk of developing one of a plurality of specified behaviors;
determining, for the first cohort of persons at a later time, how accurately the computed plurality of scores indicated the risk of developing each of the plurality of specified behaviors;
classifying at least a second person not belonging to the first cohort of persons using features of those novelty seeking associated multimodal brain networks that indicated the risk of developing the specified behaviors with greater than a predefined accuracy; and
displaying to a user the risk of the second person of developing any of the specified behaviors.

9. The system of claim 8, wherein the novelty seeking associated multimodal brain networks comprise at least one of the thalamus, the prefrontal cortex, the insular cortex, the mid temporal lobe, the striatum, the amygdala, and the hippocampus.

10. The system of claim 8, wherein the plurality of specified behaviors comprises at least some of alcohol drinking, smoking, hyperactivity, depression, and psychosis.

11. The system of claim 8, wherein the Magnetic Resonance Imaging data comprises a multiple MRI fusion, including gray matter volume (GMV) and a plurality of task-related fMRI contrasts.

12. The system of claim 9, wherein the plurality of task-related fMRI contrasts comprises at least some of a modified monetary incentive delay task, a face emotion identification task, and a stop-signal task.

13. The system of claim 8 further comprising combining the features of those novelty seeking associated multimodal brain networks that indicated the risk of developing the specified behaviors with greater than a predefined accuracy to form a model of a generalized dysfunctional novelty seeking associated multimodal brain network.

14. The system of claim 8, wherein classifying the risk of developing any of the plurality of specified behaviors comprises at least one of: a regression model, a machine learning model, an image analysis, a support vector machine, a binary classification, or a multi-class classification.

15. A computer program product comprising a non-transitory computer readable storage having program instructions embodied therewith, the program instructions executable by a computer, to cause the computer to perform a method comprising:
identifying a highest portion of novelty seeking associated multimodal brain networks in a first cohort of persons based on Magnetic Resonance Imaging data;
computing a plurality of scores, each score indicating a risk of developing one of a plurality of specified behaviors;
determining, for the first cohort of persons at a later time, how accurately the computed plurality of scores indicated the risk of developing each of the plurality of specified behaviors;
classifying at least a second person not belonging to the first cohort of persons using features of those novelty seeking associated multimodal brain networks that indicated the risk of developing the specified behaviors with greater than a predefined accuracy; and
displaying to a user the risk of the second person of developing any of the specified behaviors.

16. The computer program product of claim 15, wherein the novelty seeking associated multimodal brain networks comprise at least one of the thalamus, the prefrontal cortex, the insular cortex, the mid temporal lobe, the striatum, the amygdala, and the hippocampus.

17. The computer program product of claim 15, wherein the plurality of specified behaviors comprises at least some of alcohol drinking, smoking, hyperactivity, depression, and psychosis.

18. The computer program product of claim 15, wherein the Magnetic Resonance Imaging data comprises a multiple MRI fusion, including gray matter volume (GMV) and a plurality of task-related fMRI contrasts.

19. The computer program product of claim 18, wherein the plurality of task-related fMRI contrasts comprises at least some of a modified monetary incentive delay task, a face emotion identification task, and a stop-signal task.

20. The computer program product of claim 15, further comprising combining the features of those novelty seeking associated multimodal brain networks that indicated the risk of developing the specified behaviors with greater than a predefined accuracy to form a model of a generalized dysfunctional novelty seeking associated multimodal brain network and, wherein classifying the risk of developing any of the plurality of specified behaviors comprises at least one of: a regression model, a machine learning model, an image analysis, a support vector machine, a binary classification, or a multi-class classification.

21. A method of determining a risk of a person developing one or more novelty seeking behaviors, implemented in a computer system comprising a processor, memory accessible by the processor, and computer program instructions stored in the memory and executable by the processor, the method comprising:
providing multimodal reward related biomarkers associated with a generalized dysfunctional novelty seeking multimodal brain network;
identifying a highest portion of novelty seeking associated multimodal brain networks in a first cohort of persons based on Magnetic Resonance Imaging data;
computing a plurality of scores, each score indicating a risk of developing one of a plurality of specified behaviors;
determining, for the first cohort of persons at a later time, how accurately the computed plurality of scores indicated the risk of developing each of the plurality of specified behaviors; and
building a predictive model able to classify the risk of a person not belonging to the first cohort of persons developing at least one of the plurality of specified behaviors based on at least magnetic resonance imaging data of the person.

22. A method, implemented in a computer system comprising a processor, memory accessible by the processor, and computer program instructions stored in the memory and executable by the processor, the method comprising:
measuring a person's brain using magnetic resonance imaging;
feeding the measurements into a predictive model;
determining, using the predictive model, a person's risk of developing one or more of a plurality of specified behaviors; and
displaying to a user the determined risk that the person is likely to develop one or more of the plurality of specified behaviors.
